(19) United States
(12) Patent Application Publication
(10) Pub. No.: US 2025/0011816 A1
(43) Pub. Date: Jan. 9, 2025

(54) FEL D 1 KNOCKOUTS AND ASSOCIATED COMPOSITIONS AND METHODS BASED ON CRISPR-CAS9 GENOMIC EDITING

(71) Applicant: Indoor Biotechnologies Inc., Charlottesville, VA (US)

(72) Inventors: Martin D. Chapman, Charlottesville, VA (US); Olya Spassibojko, Marlboro, NJ (US)

(21) Appl. No.: 18/680,548

(22) Filed: May 31, 2024

Related U.S. Application Data
(63) Continuation of application No. 16/082,157, filed on Sep. 4, 2018, now Pat. No. 12,037,601.
(60) Provisional application No. 62/303,686, filed on Mar. 4, 2016.

(51) Int. Cl. C12N 15/90; A01K 67/0276; C12N 15/11

(52) U.S. Cl. CPC C12N 15/907; A01K 67/0276; C12N 15/11; A01K 2217/075; A01K 2227/10; A01K 2267/02; C12N 2310/20; C12N 2800/80

(57) ABSTRACT

Compositions and methods for genetically modifying felines or feline cells are described. The compositions and methods are useful for knocking out all or a portion of a Fel d 1 gene from a feline genome. Feline cells and organisms in which all or a portion of the Fel d 1 gene is knocked out are also described. The compositions and methods may include reagents and procedures for CRISPR-Cas9-mediated genomic editing of Fel d 1.

### CROSS-REFERENCE TO RELATED APPLICATIONS

This application is a continuation application of U.S. patent application Ser. No. 16/082,157 filed Sep. 4, 2018. The '157 application is a National Stage application under 35 USC § 371 of International Application No. PCT/US17/20609, filed Mar. 3, 2017, which application relies on the disclosure of and claims priority to and the benefit of the filing date of U.S. Provisional Application No. 62/303,686, filed Mar. 4, 2016, the disclosures of each of which are hereby incorporated by reference herein in their entireties.

### SEQUENCE LISTING

The instant application contains a Sequence Listing which has been submitted electronically in XML file format and is hereby incorporated by reference in its entirety. Said XML copy, created on Apr. 25, 2024, is named WR-IB-101-CON_SL.xml and is 2,188,101 bytes in size.

### BACKGROUND OF THE INVENTION

### Field of the Invention

The present invention generally relates to compositions and methods for genetically modifying felines or feline cells. More particularly, the present invention relates to compositions and methods for knocking out all or a portion of a Fel d 1 gene from a feline genome, as well as feline cells and organisms in which all or a portion of the Fel d 1 gene is knocked out.

### Description of Related Art

Allergy and asthma have become increasingly prevalent in modern society. Common allergens come from a variety of different sources, ranging from dust mites to foods. Household pets are another typical source of allergens, and some (e.g. cat and dog) are commonly found in homes without pets. However, household pets play a significant role in people's daily lives, providing company and comfort to a variety of individuals of all ages and backgrounds, many of whom are allergic to their pets but could not bear to give them up. Cat allergens, in particular, are a common health concern for numerous patients, and the allergens are essentially ubiquitous in society. Particles of cat allergens readily become airborne and remain for hours, and the allergen also sticks to surfaces such as fabrics, resulting in its transmission from a cat-owning household to the rest of the world. Significant levels of cat allergens can be found in offices, stores, or schools, regardless of whether a cat has been present in that environment, due to such high transmission rates of the allergens.

Allergy to cats affects 10-15% of adults and 1 out of 7 children between the ages of 6-19. Symptoms of patients suffering from cat allergy may range from rhinoconjunctivitis to severe asthma. Though multiple cat allergens have been identified, up to 95% of patients have IgE antibodies specific to the major allergen from cat, Fel d 1. Fel d 1 is recognized by 60-90% of an allergic individual's total IgE antibodies.

Fel d 1 is a protein of the secretoglobin family and exists as a tetramer that is 35 kD in molecular weight. The tetrameric protein is composed of two heterodimers, each of which consists of two antiparallel chains linked by three disulfide bonds, each encoded by a different gene. See Grönlund et al., Formation of Disulfide Bonds and Homodimers of the Major Cat Allergen Fel d 1 Equivalent to the Natural Allergen by Expression in Escherichia coli, J. Biol. Chem., Vol. 278, No. 41, pp. 40144-40141 (Oct. 10, 2003). The gene encoding chain 1 (for which the protein product is 70 amino acids (8 kD)) and chain 2 (92 amino acids (10 kD)) are arranged symmetrically in the feline genome, starting in a narrow region and extending outwards, encoded by opposite strands of the DNA (see Grönlund et al., The Major Cat Allergen, Fel d 1, in Diagnosis and Therapy, Int Arch Allergy Immunol (2010), 151:265-274 and Morgenstern, et al., Amino acid sequence of Fel d 1, the major allergen of the domestic cat: Protein sequence analysis and cDNA cloning, Proc Natl Acad Sci USA, 88 (21): 9690-9694 (1991). The two genes are located in a genomic span of less than 10,000 base pairs.

Analysis of sequence and structural homology suggests that Fel d 1 is most closely related to Androgen-Binding Proteins (especially those of the mouse and yak) and other uteroglobin allergens (such as those produced by bats and rabbits). The Fel d 1 protein binds Ca²⁺ ions, and based on its structure, also contains two internal cavities that could bind steroid ligands. Additionally, it has been hypothesized that Fel d 1 may play a role in protecting the epithelium or have anti-inflammatory properties. However, despite years of studying Fel d 1, the biological function of the Fel d 1 protein remains unknown.

In the cat, Fel d 1 is produced by the sebaceous glands of the skin, as well as by the salivary, perianal, and lachrymal glands. Fel d 1 is transferred to the fur from saliva, through grooming, and from skin. Kittens are known to produce less Fel d 1 than adult cats, and females produce lower levels of Fel d 1 compared to males. Neutered cats also produce significantly less Fel d 1 than unaltered cats. When dried Fel d 1 particles on dander and cat hair become airborne, they can remain airborne for hours. Humans come in contact with Fel d 1 either by inhalation of these particles, or through direct contact with a cat. The immune system response to Fel d 1 is IgE-antibody mediated. When the Fel d 1 protein binds to receptors on immune cells, it results in the release of pro-inflammatory agents including cytokines and histamines, which stimulate the allergy symptoms. There is also evidence that Fel d 1 has gelatin-and fibronectin-degrading activity and, like other uteroglobins, is an inhibitor to the activity of phospholipase A2, but these additional properties have not been directly linked to the immune response.

Attempts to mitigate the amount of Fel d 1 present in the environment of cat-allergic patients have included limiting cats' access to bedrooms, frequent vacuuming or steam cleaning, and even regular cat washing. Additionally, the use of High-Efficiency Particulate Air (HEPA) filters has been shown to decrease the amount of airborne allergen present in the environment. Still, these methods cannot completely eliminate Fel d 1, and symptoms of severely allergic patients may persist, often for a long time after the cat has been in the environment due to trace amounts of allergen remaining.

Some individuals allergic to cats opt to search for animals specifically tested and proven to produce lower levels of Fel d 1, but selective breeding can only reduce an animal's allergens by so much, and perhaps not enough for severely allergic patients. Further, animals selectively bred for low allergen levels are extremely time-consuming and costly to produce. Additionally, extensive in-breeding of animal lineages can result in increased coincidence of rare genetic mutations, and consequently, severe genetic disease. So, while the practice of selective breeding may be practical in the short-term, it is unsustainable as a long-term solution to generating hypoallergenic animals.

Related efforts in this area include those described in U.S. Patent Application Publication Nos. 2003/0177512 and 2011/0023156, each incorporated by reference in their entireties. Yet, there remains a need in the art for improved methods and compositions that address these issues.

### SUMMARY OF THE INVENTION

The present invention provides compositions and methods for selective deletion of one or more Fel d 1 genes in a cell of a cat, such as a domestic house cat (Felis catus). The one or more Fel d 1 genes may include chain 1, chain 2, or both chain 1 and chain 2.

In embodiments, the present invention provides compositions and methods for targeting a Fel d 1 genomic sequence in a cat cell. In one aspect of this embodiment, the invention provides a plurality of polynucleotide sequences that can be used in a CRISPR-Cas9 system to target Fel d 1 for genome editing. The polynucleotides may include any sgRNA that may have utility in a CRISPR-Cas9 system for targeted deletion of Fel d 1 or a portion thereof from the genome of a cat cell.

Embodiments of the invention provide a chimeric guide RNA (sgRNA) comprising a first polynucleotide and a second polynucleotide. The first polynucleotide is a crRNA. The second polynucleotide is a tracrRNA. The crRNA (first polynucleotide) is selected such that it is capable of binding to a portion of one strand of DNA located in a target DNA region. The complement of that strand is referred to herein as the sgRNA Target Sequence, which is identical in sequence to the crRNA. The sgRNA Target Sequence may also be referred to herein as the crRNA target sequence since it is the crRNA portion of the sgRNA that directs the sgRNA to the target DNA site. As used herein, “identical in sequence” means that the crRNA has the same sequence as the sgRNA Target Sequence, but that the crRNA contains uracil (U) in place of thymine (T). The sgRNA Target Sequence is a portion of a Fel d 1 genomic sequence or a flanking region, wherein the portion is 5′ of a PAM sequence consisting of NGG, wherein N=A, T, C, or G.

In embodiments, the first polynucleotide of the sgRNA (crRNA) is identical in sequence to a portion of Fel d 1 chain 1 genomic sequence or 3′ flanking region 1 kb in length or a portion of Fel d 1 chain 2 or a 5′ flanking region 1 kb in length.

In embodiments, the first polynucleotide of the sgRNA (crRNA) is identical in sequence to a portion of the sequence set forth in SEQ ID NO: 1226 or SEQ ID NO: 1227, or is substantially complementary to a portion of the sequence set forth in SEQ ID NO: 1226 or SEQ ID NO: 1227.

In embodiments, the first polynucleotide of the sgRNA (crRNA) is 18-22 nucleotides in length, such as 18, 19, 20, 21, or 22 nucleotides in length.

In embodiments, the first polynucleotide of the sgRNA (crRNA) is identical in sequence to a DNA sequence set forth in SEQ ID NOS: 1-1225 (exemplary sgRNA Target Sequences).

In embodiments, the first polynucleotide of the sgRNA is a crRNA that is at least 60%, 65%, 70%, 75%, 80%, 85%, 90%, 95%, 96%, 97%, 98%, or 99% identical in sequence to a DNA sequence set forth in SEQ ID NOS: 1-1225.

In embodiments, the first polynucleotide of the sgRNA (crRNA) is located 5′ of the second polynucleotide of the sgRNA (tracrRNA).

Embodiments of the invention include a chimeric DNA molecule encoding any chimeric guide RNA described herein.

In embodiments, the chimeric DNA molecule comprises a first polynucleotide and a second polynucleotide. The first polynucleotide of the chimeric DNA molecule is identical to a portion of a Fel d 1 genomic sequence or a flanking region, wherein the portion is 5′ of a PAM sequence consisting of NGG, wherein N=A, T, C, or G. The second polynucleotide encodes a tracrRNA.

In embodiments, the first polynucleotide of the chimeric DNA molecule is identical to a portion of Fel d 1 chain 1 genomic sequence or 3′ flanking region 1 kb in length or a portion of Fel d 1 chain 2 or a 5′ flanking region 1 kb in length.

In embodiments, the first polynucleotide of the chimeric DNA molecule is identical to a portion of the sequence set forth in SEQ ID NO: 1226 or SEQ ID NO: 1227, or is substantially complementary to a portion of the sequence set forth in SEQ ID NO: 1226 or SEQ ID NO: 1227.

In embodiments, the first polynucleotide of the chimeric DNA molecule is identical to a DNA sequence set forth in SEQ ID NOS: 1-1225.

In embodiments, the first polynucleotide of the chimeric DNA molecule is at least 60%, 65%, 70%, 75%, 80%, 85%, 90%, 95%, 96%, 97%, 98%, or 99% identical to a DNA sequence set forth in SEQ ID NOS: 1-1225.

In embodiments, the second polynucleotide of the chimeric DNA molecule comprises a sequence set forth in SEQ ID NOS: 1228-1233 or a fragment thereof, and the second polynucleotide of the sgRNA (tracrRNA) is selected to be identical in sequence to a DNA sequence set forth in SEQ ID NOS: 1228-1233 or a fragment thereof.

Embodiments of the invention include an expression construct comprising any chimeric DNA molecule described herein, operably linked to a promoter. The promoter may be a Pol III promoter such as a U6 promoter.

Embodiments of the invention include recombinant vectors comprising any expression construct described herein.

Additionally, recombinant vectors of the invention may include a second expression construct comprising a polynucleotide encoding Cas9 operably linked to a promoter. The promoter of the second expression construct may be a constitutive mammalian promoter, an inducible promoter, or a tissue-specific promoter.

Embodiments of the invention include host cells comprising any recombinant vector described herein. In embodiments, the host cells lack expression of Fel d 1 protein or a portion thereof, and/or lack all or a portion of the genomic sequence which encodes Fel d 1 protein. In some embodiments, the host cell produces a hypoallergenic variant of Fel d 1 protein.

Embodiments of the invention include a cell line derived from any host cell described herein. In some aspects of the invention, the cell line is immortalized.

Embodiments of the invention include a method of deleting all or a portion of a Fel d 1 genomic sequence from a feline cell. The method comprises introducing at least a first and second chimeric guide RNA and a Cas9 protein into a feline cell. Each chimeric guide RNA comprises a first polynucleotide and a second polynucleotide such that the first polynucleotide is a unique crRNA that is selected to be identical in sequence to a portion of a Fel d 1 genomic sequence that is selected as an sgRNA target sequence and the second polynucleotide is a tracrRNA. The first chimeric guide RNA and second chimeric guide RNA direct the Cas9 protein to produce a first and second double stranded break in a portion of Fel d 1 genomic sequence or a flanking region such that upon repair of the first and second double stranded breaks, an intervening portion comprising a Fel d 1 genomic sequence is removed from the genome of the feline cell.

In embodiments, the crRNA of the first chimeric guide RNA is identical in sequence to a portion of a Fel d 1 genomic sequence that is located 5′ of Fel d 1 chain 2 genomic sequence and the crRNA of the second chimeric guide RNA is identical in sequence to a Fel d 1 genomic sequence that is located 3′ of Fel d 1 chain 1.

In embodiments, the crRNA of the first chimeric guide RNA is identical in sequence to a portion of a Fel d 1 genomic sequence that is located internal to a portion of a Fel d 1 chain 2 genomic sequence and the crRNA of the second chimeric guide RNA is identical in sequence to a Fel d 1 genomic sequence that is located internal to Fel d 1 chain 1 genomic sequence.

In embodiments, the crRNA of the first chimeric guide RNA is identical in sequence to a portion of a Fel d 1 genomic sequence that is located 5′ of Fel d 1 chain 2 genomic sequence and the crRNA of the second chimeric guide RNA is identical in sequence to a portion of a Fel d 1 genomic sequence that is located internal to Fel d 1 chain 1 genomic sequence.

In embodiments, the crRNA of the first chimeric guide RNA is identical in sequence to a portion of a Fel d 1 genomic sequence that is located internal to Fel d 1 chain 2 genomic sequence and the crRNA of the second chimeric guide RNA is identical in sequence to a portion of a Fel d 1 genomic sequence that is located 3′ of Fel d 1 chain 1 genomic sequence.

In embodiments, the portion of the Fel d 1 genomic sequences are chosen from the DNA sequences set forth in SEQ ID NOS: 1-1225.

Additional embodiments include a method of deleting all or a portion of a Fel d 1 genomic sequence from a cell. The method comprises introducing a first and second pair of chimeric guide RNAs (sgRNAs) and a Cas9 nickase into a feline cell. Each chimeric guide RNA comprises a first polynucleotide and a second polynucleotide such that the first polynucleotide is a unique crRNA identical in sequence to a portion of a Fel d 1 genomic sequence selected as an sgRNA target sequence and the second polynucleotide is a tracrRNA. The first pair of chimeric guide RNAs direct the Cas9 nickase to produce a first pair of single stranded breaks in a first portion of Fel d 1 genomic sequence or a flanking region and the second pair of chimeric guide RNAs direct the Cas9 nickase to produce a second pair of single stranded breaks in a second portion of Fel d 1 genomic sequence or a flanking region such that upon repair of the first and second pair of single stranded breaks, an intervening portion comprising a Fel d 1 genomic sequence is removed from the genome of the feline cell.

In embodiments, the first pair of chimeric guide RNAs are selected to be capable of base pairing with a genomic sequence that is located 5′ of Fel d 1 chain 2 genomic sequence and the second pair of chimeric guide RNAs are selected to be capable of base pairing with a genomic sequence that is located 3′ of Fel d 1 chain 1 genomic sequence.

In embodiments, the first pair of chimeric guide RNAs are selected to be capable of base pairing with a genomic sequence that is located internal to Fel d 1 chain 2 genomic sequence and the second pair of chimeric guide RNAs are selected to be capable of base pairing with a genomic sequence that is located internal to Fel d 1 chain 1 genomic sequence.

In embodiments, the first pair of chimeric guide RNAs are selected to be capable of base pairing with a genomic sequence that is located 5′ of Fel d 1 chain 2 genomic sequence and the second pair of chimeric guide RNAs are selected to be capable of base pairing with a genomic sequence that is located internal to Fel d 1 chain 1 genomic sequence.

In embodiments, the first pair of chimeric guide RNAs are selected to be capable of base pairing with a genomic sequence that is located internal to Fel d 1 chain 2 genomic sequence and the second pair of chimeric guide RNAs are selected to be capable of base pairing with a genomic sequence that is located 3′ of Fel d 1 chain 1 genomic sequence.

In embodiments, the portion of the Fel d 1 genomic sequence selected as the sgRNA Target Sequence is chosen from the DNA sequences set forth in SEQ ID NOS: 1-1225. In embodiments, the sgRNA Target Sequence is the complement of the portion of the Fel d 1 genomic sequence that the chimeric guide RNAs are selected to be capable of base pairing with.

In embodiments of the methods described herein, chimeric guide RNAs and Cas9 protein are introduced directly into the cell as ribonucleoprotein complexes.

In embodiments of the methods described herein, chimeric guide RNAs and Cas9 protein are introduced indirectly into the cell by way of one or more expression cassettes.

In embodiments, the first and second chimeric guide RNAs and Cas9 protein are introduced indirectly into the cell by way of one or more vectors.

Embodiments include a method of treating a cat, such as administering a recombinant vector to a cat that is wild-type for Fel d 1 expression. The recombinant vector encodes at least a first and second chimeric guide RNA and a Cas9 protein. Each chimeric guide RNA (sgRNA) comprises a first polynucleotide and a second polynucleotide such that the first polynucleotide is a unique crRNA that is selected to be encoded by a portion of a Fel d 1 genomic sequence selected as an sgRNA target sequence and the second polynucleotide is a tracrRNA. The first chimeric guide RNA and second chimeric guide RNA direct the Cas9 protein to produce a first and second double stranded break in a portion of Fel d 1 genomic sequence or a flanking region such that upon repair of the first and second double stranded breaks, an intervening portion comprising a Fel d 1 genomic sequence is removed from the genome.

In embodiments, the recombinant vector is a viral vector such as a lentiviral vector. Further, in embodiments, the recombinant vector comprises a tissue-specific or inducible promoter driving Cas9 expression.

Further, the recombinant vector can be administered systemically, or to specific tissues such as salivary gland or skin tissue.

Further, in embodiments of methods of the invention, Fel d 1 protein expression is impaired or absent in skin, salivary glands, perianal glands, or lachrymal glands.

Additional embodiments of the invention include methods of producing a cat in which Fel d 1 expression is impaired or absent.

In one embodiment, the method comprises culturing any of the cell lines or host cells described herein, placing a single engineered cell or host cell into an enucleated ovum to create a cloned embryo, implanting the cloned embryo into a recipient female cat, and allowing the cloned embryo to mature into a cat.

In another embodiment, the method comprises culturing a cat oocyte, introducing into the cat oocyte a pair of guide RNAs and the Cas9 protein, wherein the guide RNAs are designed to be encoded by a Fel d 1 genomic sequence of a 5′ or 3′ flanking region, fertilizing the oocyte with cat sperm to create an embryo, culturing the embryo in vitro, implanting the embryo into a recipient female cat, and allowing the embryo to mature into a cat.

Additional embodiments include transgenic or knock-out cats produced according to any method described herein.

An embodiment of the invention includes a cat lacking a portion of a Fel d 1 genomic sequence which results in impaired or absent expression of Fel d 1 protein. In embodiments, the Fel d 1 protein or a portion thereof is expressed but is hypoallergenic in humans.

In some embodiments, all or a portion of the Fel d 1 genomic sequence is deleted from somatic cells. In some embodiments, all or a portion of the Fel d 1 genomic sequence is deleted from germline cells. In some embodiments, all or a portion of the Fel d 1 genomic sequence is deleted from both somatic cells and germline cells. In some embodiments, at least 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 kb of a Fel d 1 genomic sequence, including 5′ and or 3′ flanking regions, is deleted.

Another embodiment of the invention includes F1 progeny of a male and female cat embodiment described herein.

### BRIEF DESCRIPTION OF THE DRAWINGS

The accompanying drawings illustrate certain aspects of embodiments of the present invention and should not be used to limit the invention. Together with the written description the drawings serve to explain certain principles of the invention.

FIG. 1 is a genomic map of Felis catus Fel d genomic region, chains 1 and 2.

FIG. 2A is a genomic map of Felis catus Fel d 1 chain 1.

FIG. 2B is a genomic map of Felis catus Fel d 1 chain 2.

FIG. 3 is a genomic map of Felis catus Fel d genomic region, chains 1 and 2, showing exemplary sgRNA target regions.

FIG. 4A is an outline of a strategy for designing sgRNAs for use with Cas9 according to an embodiment of the invention.

FIG. 4B is an outline of a strategy for designing sgRNAs for use with Cas9 nickase according to an embodiment of the invention.

FIG. 5A is a schematic diagram of a guide RNA expression cassette according to an embodiment of the invention.

FIG. 5B is a schematic diagram of a Cas9 expression cassette according to an embodiment of the invention.

FIG. 6A is a schematic diagram of a vector comprising a cloning site for insertion of an oligonucleotide encoding a crRNA upstream of a polynucleotide encoding a tracrRNA according to an embodiment of the invention.

FIG. 6B is a schematic diagram of the vector of FIG. 6A with a crRNA cloned into the vector according to an embodiment of the invention.

FIG. 7 is a schematic diagram of a vector comprising a Cas9 expression cassette according to an embodiment of the invention.

FIG. 8A is a schematic diagram of a vector comprising two guide RNA expression cassettes according to an embodiment of the invention.

FIG. 8B is a schematic diagram of a vector comprising up to 8 guide RNA expression cassettes according to an embodiment of the invention.

FIG. 9A is a schematic diagram of a vector comprising a guide RNA expression cassette and a Cas9 expression cassette according to embodiments of the invention.

FIG. 9B is a schematic diagram of a vector comprising a two guide RNA expression cassettes and a Cas9 expression cassette according to embodiments of the invention.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

The present invention provides compositions and methods for selective deletion of genomic DNA encoding one or more Fel d 1 genes in cat cells and ultimately in a living cat. The composition and methods are useful for precise genomic engineering to selectively eliminate production of Fel d 1, resulting in a domestic cat that no longer produces this key allergen.

Embodiments of methodologies of the present invention include those based on CRISPR-Cas9 mediated gene editing, which is known in the art (see Cong et al., Multiplex Genome Engineering Using CRISPR/Cas Systems, Science 15 Feb. 2013: 339 (6121), 819-823 “Cong et al., 2013”; Ran et al., Genome engineering using the CRISPR-Cas9 system, Nat Protoc. 2013 Nov. 8 (11): 2281-2308 (“Ran et al., 2013”); Doudna J A and Charpentier E, The new frontier of genome engineering with CRISPR-Cas9, Science, 2014, 346:6213, 1258096-1-9; and Jinek et al., A Programmable Dual-RNA-Guided DNA Endonuclease in Adaptive Bacterial Immunity, Science 337, 816 (2012) (“Jinek, 2012”)). Briefly, CRISPR is an RNA-guided genomic editing system that employs a bacterially derived protein (Cas9) and a synthetic guide RNA to introduce a double strand break at a specific location within the genome. By transfecting a cell with the Cas9 protein along with a specially designed guide RNA (gRNA, or sgRNA) that directs the cut through hybridization with its matching genomic sequence, site-specific cleavage of genomic DNA can be introduced into a cell. Upon repair of the double-strand break by the cell's DNA repair systems, errors can be introduced to generate a knockout of a particular gene. Additionally, an engineered Cas9 protein (Cas9 “nickase”) functions similarly except it introduces single-stranded breaks instead of double-stranded breaks.

The details of the backbone vectors, other reagents for SpCas9 genome engineering system, and protocols can be found in the Cong and Ran publications referenced above, as well as the website for Dr. Feng Zhang at the Broad Institute (http://www.genome-engineering.org/crispr/). Features of the system such as the amino acid sequence of Streptococcus pyogenes Cas9 (for example, see public Accession No. Q99ZW2 (NCBI)) and sequence of the tracrRNA (see Jinek, 2012) are publicly available. Exemplary DNA sequences encoding Streptococcus pyogenes tracrRNA or fragments thereof are set forth in SEQ ID NOS: 1228-1233. Reagents, cloning vectors, and kits for guide RNA and Cas9 expression and associated assays are available from commercial vendors such as GenScript, BioRad, Thermo-Fisher, Sigma-Aldrich, OriGene, and Clontech. Additionally, CRISPR plasmids (including those from the Zhang lab) have been deposited at and are available from Addgene (Cambridge, MA), a non-profit plasmid repository, and a number of protocols and resources are available on the Addgene website (http://www.addgene.org/crispr/).

In addition, background information on CRISPR-mediated gene editing can be found in U.S. Pat. Nos. 8,697,359; 8,795,965; 8,865,406; 8,871,445; 8,889,356; 8,889,418; 8,906,616; 8,932,814; 8,945,839; 8,993,233; 8,999,641 as well as U.S. Patent Application Publication Nos. 2014/0068797; 2014/0170753; 2014/0179006; 2014/0179770; 2014/0186843; 2014/0186919; 2014/0186958; 2014/0189896; 2014/0227787; 2014/0234972; 2014/0242664; 2014/0242699; 2014/0242700; 2014/0248702; 2014/0256046; 2014/0273231; 2014/0273232; 2014/0273234; 2014/0287938; 2014/0310830; 2014/0335620; 2014/0357530; 2015/0020223; 2015/0031134; 2015/0079681; 2015/0184139; 2015/0203872; 2015/0232882; 2015/0232883; 2015/0247150; 2015/0291965; 2015/0291966; 2015/0356239; the disclosures of these patents and published applications are hereby incorporated by reference herein in their entireties.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

In aspects of the invention the terms “guide RNA”, “single guide RNA”, “synthetic guide RNA”, “sgRNA”, and “chimeric guide RNA” are used interchangeably and refer to a chimeric polynucleotide sequence comprising a “guide sequence” and a “tracr sequence”.

The term “guide sequence” may be used interchangeably with the terms “guide” or “CRISPR-targeting RNA” or “crRNA”. In general, the guide sequence refers to the polynucleotide sequence within the guide RNA that is selected to hybridize with a corresponding DNA molecule at a specific location and is capable of directing sequence-specific binding of a CRISPR complex at that target location within the DNA molecule. In some embodiments, the degree of complementarity between a guide sequence and the corresponding sequence to which it will bind, when optimally aligned using a suitable alignment algorithm, is about or more than about 50%, 60%, 75%, 80%, 85%, 90%, 95%, 97.5%, 99%, or more. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In some embodiments, the guide sequence is about 5 to 40, 10 to 30, 15 to 25, 16 to 24, 17 to 23, 18 to 22, or 19 to 21, or 20 nucleotides in length. Preferably, the guide sequence is 18-22 nucleotides in length. The ability of a guide sequence to direct sequence-specific binding of a CRISPR complex at a target DNA location may be assessed by any suitable assay.

The term “tracrRNA” or “tracr sequence” refers to a portion of the guide RNA that acts as a scaffold between the crRNA and Cas9 endonuclease. Exemplary polynucleotides encoding tracrRNA are set forth in SEQ ID NOS: 1228-1233. Thus, suitable tracrRNA have a polynucleotide sequence that is identical in sequence to the polynucleotides in SEQ ID NOS: 1228-1233 or a fragment thereof. In some embodiments, the tracrRNA, or polynucleotides encoding tracrRNA may be about 50, 55, 60, 65, 70, 75, 80, 85, 90, 95, or 100 nucleotides in length.

The term “CRISPR” refers to Clustered Regularly Interspaced Short Palindromic Repeats, a series of short direct repeats in the genome of select bacteria and archaea that are interspaced with short spacer sequences of plasmid or viral origins.

The terms “CRISPR complex” and “CRISPR-Cas” refer to a specific system of adaptive immunity in select bacteria and archaea mediated by RNA and nucleases which enable the organisms to respond to and eliminate invading genetic material.

The term “Cas9” or “Cas9 endonuclease” (also known as COG3513, Csx12, Cas5, or Csn1) refers to a CRISPR-associated protein with two nuclease domains that uses a crRNA: tracrRNA duplex for site-specific double-stranded cleavage of DNA. In aspects of the invention, the terms may refer to a wild-type Cas9 protein, or any variant, including mutants, homologs, orthologs, that mediate RNA-guided double-stranded or single-stranded cleavage of DNA.

The term “Cas9 nickase” refers to a Cas9 endonuclease that is engineered so that one of its nuclease domains is non-functional so that its activity is limited to single-stranded cleavage of DNA.

The term “target sequence” refers to a specific region of DNA (such as a genomic DNA sequence) that is the target of a guide RNA. A DNA strand within this region hybridizes with the crRNA portion of the guide RNA. The sgRNA Target Sequence is a polynucleotide sequence located in this target DNA region. In embodiments, the crRNA is selected to hybridize (otherwise referred to as binding or base pairing) with the polynucleotide sequence that is the complement of the sgRNA Target Sequence.

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature. “Variant” DNA molecules are DNA molecules containing minor changes in a native sequence, i.e., changes in which one or more nucleotides of a native sequence is deleted, added, and/or substituted. Variant DNA molecules can be produced, for example, by standard DNA mutagenesis techniques or by chemically synthesizing the variant DNA molecule or a portion thereof. Such variants preferably do not change the reading frame of the protein-coding region of the polynucleotide and can encode a protein having no change, a reduction, or an increase in a desired biological activity. “Variant” peptides or proteins are those which include changes in the amino acid sequence include substitutions, deletions, and/or insertions.

The terms “non-naturally occurring” or “engineered” or “genetically-engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions. According to certain aspects, any portion of the Fel d 1 gene which results in disruption of Fel d 1 protein synthesis may be targeted by the compositions and methods of the invention. As understood herein, a “Fel d 1 gene” can be used interchangeably with a “Fel d 1 genomic sequence”.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, “identity” is a measure of the identity of nucleotide sequences or amino acid sequences compared to a reference nucleotide or amino acid sequence. In general, the sequences are aligned so that the highest order match is obtained. “Identity” per se has an art-recognized meaning and can be calculated using published techniques. (Sec, e.g., Computational Molecular Biology, Lesk, A. M., ed., Oxford University Press, New York (1988); Biocomputing: Informatics And Genome Projects, Smith, D. W., ed., Academic Press, New York (1993); Computer Analysis of Sequence Data, Part I, Griffin, A. M., and Griffin, H. G., eds., Humana Press, New Jersey (1994); von Heinje, G., Sequence Analysis In Molecular Biology, Academic Press (1987); and Sequence Analysis Primer, Gribskov, M. and Devereux, J., eds., M Stockton Press, New York (1991)). While there exists several methods to measure identity between two polynucleotide or polypeptide sequences, the term “identity” is well known to skilled artisans (Carillo, H. & Lipton, D., Siam J Applied Math 48:1073 (1988)). Methods commonly employed to determine identity or similarity between two sequences include, but are not limited to, those disclosed in Guide to Huge Computers, Martin J. Bishop, ed., Academic Press, San Diego (1994) and Carillo, H. & Lipton, D., Siam Applied Math 48:1073 (1988). Computer programs may also contain methods and algorithms that calculate identity and similarity. Examples of computer program methods to determine identity and similarity between two sequences include, but are not limited to, GCG program package (Devereux, J., et al., Nucleic Acids Research 12 (i): 387 (1984)), BLASTP, ExPASy, BLASTN, FASTA (Atschul, S. F., et al., J Molec Biol 215:403 (1990)) and FASTDB. Examples of methods to determine identity and similarity are discussed in Michaels, G. and Garian, R., Current Protocols in Protein Science, Vol 1, John Wiley & Sons, Inc. (2000), which is incorporated by reference. In one embodiment of the present invention, the algorithm used to determine identity between two or more polypeptides is BLASTP.

As used herein, a sequence is “substantially identical” if it at least 60%, 65%, 70%, 75%, 80%, 85%, 90%, 95%, 97%, 98%, 99%, or 100% identical to a reference sequence.

“Operably Linked.” A first nucleic-acid sequence is “operably linked” with a second nucleic-acid sequence when the first nucleic-acid sequence is placed in a functional relationship with the second nucleic-acid sequence. For instance, a promoter is operably linked to a coding sequence if the promoter affects the transcription or expression of the coding sequence. Generally, operably linked DNA sequences are contiguous and, where necessary to join two protein coding regions, in reading frame.

A cell, tissue, organ, or organism into which has been introduced a foreign polynucleotide, such as a recombinant vector, is considered “transformed”, “transfected”, or “transgenic.” A “transgenic” or “transformed” cell or organism also includes progeny of the cell or organism.

As used herein, “knockout” refers to a cell or organism in which a gene product is eliminated or any procedure resulting in such elimination. In certain aspects of the invention, a “knockout” can result from insertions, deletions, substitutions, frameshift mutations, and the like, that result from the compositions and methods described herein. As used herein, “knockout” may also refer to a genetically-engineered cell or organism in which a portion of DNA (such as genomic DNA) is removed. The term “knockout” as it refers to a gene is intended to mean removal or all or a portion of the DNA associated with that gene. In certain aspects, the compositions and methods of the invention have utility for knocking out all or a portion of a Fel d 1 gene.

As used herein, “hypoallergenic” means designed to reduce or minimize the possibility of an allergic response. In certain aspects of the invention, a hypoallergenic cat may result from “knockout” of the cat allergen Fel d 1. However, this invention contemplates that the compositions and methods may produce Fel d 1 hypoallergenicity through a variety of mechanisms, including any change in Fel d 1 two-dimensional or three-dimensional structure that results in reduced IgE reactivity.

Compositions of the invention include one or more guide RNAs and one or more DNA sequences encoding guide RNAs that may direct site-specific cleavage of a Fel d 1 genomic sequence, or any flanking portion that is 5′ or 3′ of the Fel d 1 genomic sequence. By identifying appropriate target sequences in the cat genome, guide RNAs or DNA sequences encoding guide RNAs may be identified. Candidate guide RNA target sequences may be identified from published genomic data or by isolating genomic DNA from a feline tissue or cell sample and sequencing the genomic DNA. In an embodiment, SEQ ID NOS: 1-1225 represent 1225 candidate target sequences in the cat genome which may be the site of site-specific double stranded cleavage of a Fel d 1 genomic sequence or any adjacent 5′ or 3′ flanking region. The candidate target sequences may be identified in silico through one or more algorithms implemented by a computer processor, or manually.

Methods of the invention include, in embodiments, screening guide RNAs for efficacy in initiating genomic editing of Fel d 1 through in vitro techniques, which may include high-throughput screening. Upon identification of guide RNAs having efficacy in vitro, active guide RNAs can be introduced into a host cell, and the engineered host cell can be used as a basis for cloning a cat that lacks expression of the Fel d 1 protein.

Methods of the invention include cloning using engineered host cells as donor cells in cloning by nuclear transfer. For example, feline skin cells or adult fibroblast cells can be cultured and transfected with vectors encoding guide RNAs and Cas9. Upon confirmation of knock-out of Fel d 1, a single donor cell is placed into enucleated ova. Cloned embryos are cultured and then surgically transferred to a surrogate queen. Additionally, this invention contemplates additional methods of creating Fel d 1 knockout cells or animals, such as through direct gamete genetic modification, or manipulation of embryonic stem cells. Alternatively, viral vectors encoding guide RNAs and Cas9 can be administered to wild-type cats to excise Fel d 1 genomic sequences in vivo. The vectors can be administered systemically or to specific Fel d 1 expressing tissues.

Also included in embodiments of the invention are transgenic cats that are knockouts for Fel d 1 (or a portion thereof) as a result of method of the invention. The cats have reduced expression of Fel d 1 or a portion thereof, and may be hypoallergenic or non-allergenic for Fel d 1. The cats have Fel d 1 knocked out in somatic cells and in the germline and are thus able to pass this characteristic to progeny.

As an alternative to CRISPR-Cas9 genomic editing, the present invention contemplates the use of other methods of genomic editing, knockdown, or silencing known in the art, including knockout by homologous recombination, knockout by Cre-lox system, or knockdown by RNA interference. For example, homologous recombination involves design of a disruption construct, deletion of the Fel d 1 gene in embryonic stem cells, injection of the engineered stem cells into an embryo, implantation of embryos, screening for kittens (heterozygous Fel d 1 Δ), and breeding of heterozygotes to obtain homozygotes. Genetic engineering through a Cre-lox system involves homologous recombination to insert lox sites around an exon or full Fel d 1 gene, and then transfection and expression of Cre recombinase (see Metzger, D. & Feil, R. Engineering the mouse genome by site-specific recombination. Curr. Opin. Biotechnol. 10, 470-476). RNA interference involves introduction of siRNA to target the native cellular machinery to prevent translation or induce degradation of specific mRNAs. These techniques are known in the art and have been reviewed (see Lodish, Molecular Cell Biology, W.H. Freeman and Company, New York). Additionally, embodiments of the invention contemplate inducible knockouts, tissue-specific knockouts, genome editing using zinc finger nucleases (see U.S. Patent Application Publication No. 2011/0023156), and Cas9 variations such as epigenetic silencing (dCas9 fused to a DNA methyltransferase).

### Example: Culture of Cell Lines

Felis catus cell lines can be obtained from American Type Culture Collection (ATCC; Manassas, Virginia) and cultured according to protocols known the art, including the ATCC® Animal Cell Culture Guide available on the ATCC website. Examples of cell lines that may be available include CRFK (ATCC® CCL-94™), Fcwf-4 [Fcwf] (ATCC® CRL-2787™), FC77.T (ATCC®) CRL-6105_FL™), PG-4 (S+L−) (ATCC® CRL-2032™), MYA-1 (ATCC® CRL-2417™), FIB [FIB(N)] (ATCC® CRL-6168™), G355-5 (ATCC® CRL-2033™), Fc2Lu (ATCC® CCL-217™), FC114E.Tr (ATCC®) CRL-6167™), FC47 (ATCC®) CRL-6094™), FeT-J (ATCC® CRL-11967™), F25 (ATCC® CRL-6566™), Fc3Tg (ATCC® CCL-176™), FeLV-3281 (ATCC®) CRL-9116™), FC83.Res (ATCC® CRL-6567™), AK-D (ATCC® CCL-150™), FC2.K (ATCC® CRL-6126™), FL74-UCD-1 (ATCC® CRL-8012™), FcT-1C (ATCC® CRL-11968™).

### Example: Primary Cell Culture

Primary cell lines may be established from tissue samples or biopsies of cat tissue. Examples include bone marrow biopsies, endoscopic biopsies, skin puncture biopsies and needle biopsy procedures including fine needle aspiration, core needle biopsies, vacuum-assisted biopsies, and image-guided biopsies. These would be performed according to standard protocols used when taking a biopsy for pathology, except a portion of the tissue sample would either be immediately put in culture or cryopreserved with the use of a cryoprotectant, such as DMSO or glycerol, for later culturing. Thus, the biopsy procedures need not be elaborated here.

The cells from the portion of the biopsy may be cultured through a variety of methods known for tissue culture and primary cell culture. For example, for primary cell culture, the tissue sample may be first dissected to remove fatty and necrotic cells. Then, the tissue sample may be subject to enzymatic or mechanical disaggregation. The dispersed cells may then be incubated, and the media changed to remove loose debris and unattached cells. Because primary cells are anchorage-dependent, adherent cells, they require a surface in order to grow properly in vitro. In one embodiment, the cells are cultured in two-dimensional (2D) cultures. Typically, a plastic uncoated vessel such as a flask or petri dish is used, and the cells are bathed in a complete cell culture media, composed of a basal medium supplemented with appropriate growth factors and cytokines. During establishment of primary cultures, it may be useful to include an antibiotic in the growth medium to inhibit contamination introduced from the host tissue. Various protocols for culturing primary cells are known and a variety of resources are available, including the ATCC® Primary Cell Culture Guide, available on the American Type Culture Collection website.

### Example: Extraction of Genomic DNA

Genomic DNA can be isolated from tissue culture cells according to various protocols known in the art. For example, in one example of such a procedure, Felis catus cells are cultured in a monolayer are trypsinized and counted. The cells are pelleted by centrifuging 5 minutes at 500×g at 4 degrees, washed in ice cold PBS and re-pelleted. The cells are resuspended in 1 volume of digestion buffer (1 ml digestion buffer/10⁸ cells. Samples are incubated at 50° C. for 12-18 hours while rotating, extracted with phenol/chloroform/isoamyl alcohol, and centrifuged 10 minutes at 1700×g. The aqueous layer is transferred to a new tube, and ½ volume of 7.5 M ammonium acetate and 2 volumes of 100% ethanol may be added. The DNA is pelleted by spinning 2 minutes at 1700×g and washed with 70% ethanol; the ethanol is then decanted and allowed to air dry. The DNA is resuspended at 1 mg/ml in TE buffer. If necessary, the sample is shaken gently at room temperature to 65° C. to facilitate resuspension (˜1 g mammalian cells yields 2 mg DNA). 10% SDS is added to a final concentration of 0.1% and RNAse A to a final concentration of 1 μg/mL. The sample is incubated for 1 hour at 37° C. The sample is then phenol/chloroform extracted and ethanol precipitated.

Digestion Buffer:
100 mM NaCl
10 mM Tris [pH 8.0]
25 mM EDTA [pH 8.0]
0.5% SDS
0.1 mg/mL proteinase K

### Example: Sequencing

Genomic DNA isolated from a direct biopsy sample, cryopreserved biopsy sample, or a cultured cell sample can be subject to sequencing analysis. Various sequencing approaches are known, including Sanger (or dideoxy) method, Maxam-Gilbert, Primer Walking, and Shotgun Sequencing. Preferred are next-generation sequencing methods (also known as high-throughput sequencing), which include a number of different sequencing methods including Illumina (Solexa) sequencing, Roche 454 sequencing, Ion torrent: Proton/PGM sequencing, and SOLID sequencing. Such next-generation techniques have been reviewed in the literature (see Grada and Weinbrecht, Next-Generation Sequencing: Methodology and Application Journal of Investigative Dermatology (2013) 133, e11; and Bahassi and Stambrook, Next-generation sequencing technologies: breaking the sound barrier of human genetics, Mutagenesis, 2014 September; 29(5):303-10). Next generation sequence methods may encompass whole genome, whole exome, and partial genome or exome sequencing methods. Whole exome sequencing covers the protein-coding regions of the genome, which represents just over 1% of the genome.

### Example: Design of Guide Sequences

In embodiments, a guide sequence of the invention is selected to target one or more Fel d 1 genomic sequences. The guide sequence may target a chain 1 or chain 2 genomic sequences on either strand of genomic DNA, or any region that is 5′ or 3′ of chain 1 or chain 2. In preferred embodiments, at least one pair of guide RNAs is chosen to excise a portion of genomic DNA between the pair.

Additionally, embodiments of the invention contemplate selection of guide RNA sequences to target flanking regions of Fel d 1 sequences and/or target internal regions of Fel d 1 sequences, or both. Thus, the guide RNAs may be selected to target one or more exons, or may be selected to completely excise Fel d 1 genomic DNA. For example, in one embodiment, genomic DNA encoding one or more exons of chain 1, chain 2, or both, is targeted. In another embodiment, exon 1 of chain 1 is targeted. In another embodiment, exon 2 of chain 1 is targeted. In another embodiment, exon 3 of chain 1 is targeted. In another embodiment, exon 1 of chain 2 is targeted. In another embodiment, exon 2 of chain 2 is targeted. In another embodiment, exon 3 of chain 2is targeted. In another embodiment, multiple exons of chain 1, chain 2, or both chain 1 and chain 2 are targeted. In another embodiment, a region that is 5′ or 3′ flanking of chain 1 or chain 2 is targeted. In another embodiment, two flanking regions of Fel d 1 genes (e.g. one upstream of chain 2, another downstream of chain 1) are targeted. In embodiments of the invention, 5′ or 3′ flanking regions that are 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 kb or more in length are targeted. In another embodiment, two internal regions of Fel d 1 genes (e.g. one internal to chain 2, one internal to chain 1) are targeted. The guide RNAs may be selected to target one or more exons, or may be selected to completely excise Fel d 1 genomic DNA. In another embodiment, a guide RNA is selected to target and inactivate a regulatory element such as the promotor of a Fel d 1 genomic sequence.

Embodiments of the invention further contemplate selection of guide sequences based on predicted “on-target” activity. The guide sequences are selected to bind to one or more target sequences with minimal potential for “off-target” activity. Thus, the guide sequences should be designed to match the target sequence with minimal homology to other regions of the genome.

Additional embodiments of the invention contemplate selection of guide sequence through identification of Promoter Adjacent Motif (PAM) sequences in the genomic region to be targeted. The PAM sequences are short sequences recognized by the CRISPR complex and may vary depending on the species of Cas9. In one embodiment, the PAM sequence is specific to a S. pyogenes Cas9 and consists of the sequence NGG, wherein N is any nucleotide. The target sequences are chosen to be upstream (or 5′) of the PAM sequence.

For example, in one embodiment of a method for designing a guide sequences a PAM (NGG) sequence is identified in a Fel d 1 genomic sequence or a region flanking 5′ or 3′ of the genomic sequence. Then, a specified number of nucleotides is counted upstream (or 5′) of the PAM sequence to determine the 5′ start of the actual guide RNA sequence. In one embodiment, the start of the target sequence is 20 nucleotides upstream of the PAM sequence. Additional considerations that may increase efficacy at target sequences include a G at position 1 (from the 5′ end of the sequence) and an A or T at position 17. Additionally, it is preferred that conserved regions of Fel d 1 genomic sequence are targeted. Finally, the actual guide sequence is determined by selecting an RNA sequence that is complementary to the target sequence.

In one embodiment, the guide sequences are designed from Fel d 1 genomic sequences available in the literature or in GenBank (such as the Felis catus isolate Cinnamon breed Abyssinian chromosome E2, whole genome shotgun sequence, public Accession Number CM001392; see also Pontius, J. U., Initial sequence and comparative analysis of the cat genome, Genome Res. 17 (11), 1675-1689 (2007)). In another embodiment, the guide sequences are designed from sequencing data from one or more feline cell lines. The feline cell lines may be primary cell cultures, including those available commercially or those established from feline tissue samples.

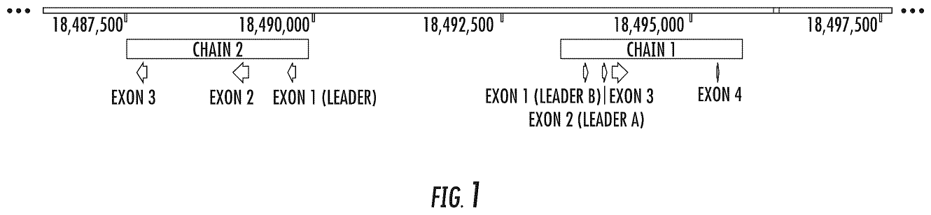

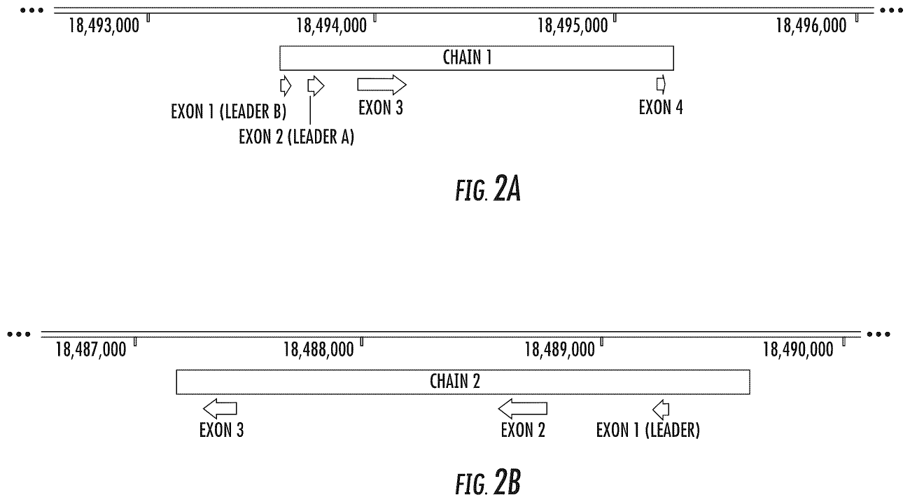

For example, FIG. 1 show a map of a 49.4 kilobase region of the Felis catus genome that comprises chain 1 and chain 2 coding regions. FIGS. 2A and 2B show a close-up map of chain 1 and chain 2 coding regions, respectively. The maps of FIGS. 1, 2A, and 2B show various features such as exons.

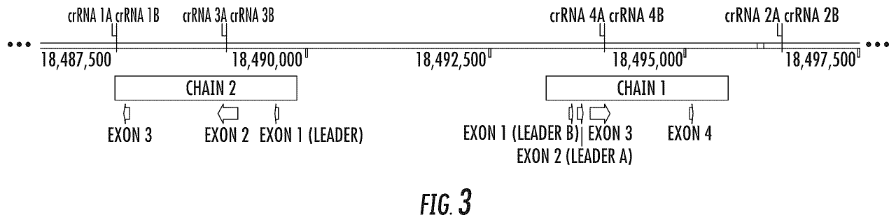

FIG. 3 shows the map of FIG. 1 with specific target regions for guide RNAs (or more particularly, the crRNA portion of the sgRNAs). In this case, eight crRNA target sites are listed (“A” representing a target site for one strand of DNA and “B” representing a corresponding target site on the complementary strand). The table below shows the relative location of the (crRNA) target sites.

TABLE 1
Exemplary crRNA Target Sites and Location

| crRNA Target Site | Location |
|---|---|
| crRNA 1A, 1B | 5′ of chain 2 |
| crRNA 2A, 2B | 3′ of chain 1 |
| crRNA 3A, 3B | internal to chain 2 |
| crRNA 4A, 4B | internal to chain 1 |

Additionally, the following table shows exemplary strategies for designing pairs of guide RNAs and in particular for selecting target portions of the Fel d 1 genomic region.

TABLE 2
Exemplary Strategies for Two-Site
Cleavage of Fel d 1 Genomic DNA

| 1^{st} site | 2^{nd} site |
|---|---|
| 5′ of chain 2 | 3′ of chain 1 |
| internal to chain 2 | internal to chain 1 |
| 5′ of chain 2 | internal to chain 1 |
| internal to chain 2 | 3′ of chain 1 |

Strategies which target as large a portion of the Fel d 1 genes as possible are preferred. For example, removing a portion of the Fel d 1 gene(s) that would in turn eliminate any potential allergic response is highly desired. One particular strategy could include choosing crRNA target sequence(s) toward the outside of the Fel d 1 region, whether the entire region is deleted or the beginning portion of the region is deleted. Thus, preferred embodiments include target sites that are 5′ and 3′ of the entire Fel d 1 genomic region, but removing any portion of the genomic region that would result in a reduced or eliminated allergic response is also possible. As an alternative strategy, beginning portions of chain 1 and chain 2 can be targeted so that neither chain is expressed.

Accordingly, pairs of crRNAs can be chosen according to the above strategy to target specific sites, such as according to the following table:

TABLE 3
Exemplary crRNA Target Sites for Cas9

| 1^{st} site | 2^{nd} site |
|---|---|
| 1A | 2B |
| 1B | 2A |
| 1A | 2A |
| 1B | 2B |
| 3A | 4B |
| 3B | 4A |
| 3A | 4A |
| 3B | 4B |
| 1A | 4B |
| 1B | 4A |
| 1A | 4A |
| 1B | 4B |
| 3A | 2B |
| 3B | 2A |
| 3A | 2A |
| 3B | 2B |

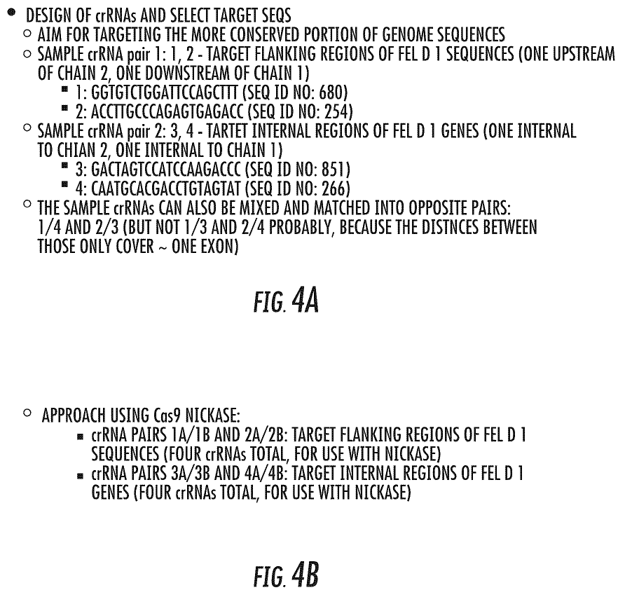

Additionally, FIG. 4A shows an exemplary strategy for selecting the crRNA portion of the guide RNAs for disruption of Fel d 1 expression.

Alternatively, if a Cas9 “nickase” is used, a total of four crRNAs would be chosen (as this would require two pairs of single-stranded breaks), such as shown in the following table. Such a strategy is also shown in FIG. 4B.

TABLE 4
Exemplary crRNA Target Sites for Cas9 Nickase

| 1^{st} site | 2^{nd} site | 3rd site | 4^{th} site |
|---|---|---|---|
| 1A | 1B | 2A | 2B |
| 3A | 3B | 4A | 4B |
| 1A | 1B | 4A | 4B |
| 3A | 3B | 2A | 2B |

However, the crRNA target sites shown in FIG. 3 are merely exemplary. The target sites can be chosen to excise a Fel d 1 genomic sequence or associated region that is anywhere from about 0.01 kb to 50 kb in length, including at least about 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, and 49 kb in length.

Table 5 shows exemplary target DNA sequences for the sites discussed above, as well as the corresponding crRNA sequences.

TABLE 5
Exemplary crRNA Target Site Sequences

| crRNA Target Site | DNA Sequence | PAM | crRNA |
|---|---|---|---|
| 1A | ggtgtctggattccagcttt | AGG | ggugucuggauuccagcuuu |
| | (SEQ ID NO: 680) | | (SEQ ID NO: 1234) |
| 1B | ctgttctttacacctaaagc | TGG | cuguucuuuacaccuaaagc |
| | (SEQ ID NO: 1166) | | (SEQ ID NO: 1235) |
| 2A | accttgcccagagtgagacc | TGG | accuugcccagagugagacc |
| | (SEQ ID NO: 254) | | (SEQ ID NO: 1236) |
| 2B | ccaagagccaggtctcactc | TGG | ccaagagccaggucucacuc |
| | (SEQ ID NO: 532) | | (SEQ ID NO: 1237) |
| 3A | gactagtccatccaagaccc | TGG | gacuaguccauccaagaccc |
| | (SEQ ID NO: 851) | | (SEQ ID NO: 1238) |
| 3B | cggactcttatccagggtct | TGG | cggacucuuauccagggucu |
| | (SEQ ID NO: 1158) | | (SEQ ID NO: 1239) |
| 4A | caatgcacgacctgtagtat | TGG | caaugcacgaccuguaguau |
| | (SEQ ID NO: 266) | | (SEQ ID NO: 1240) |
| 4B | ctggcatttgccaatactac | AGG | cuggcauuugccaauacuac |
| | (SEQ ID NO: 361) | | (SEQ ID NO: 1241) |

In embodiments, the crRNA targets are chosen to induce any appropriate mutation that results in disrupted, reduced, or eliminated expression of Fel d 1, including insertions, deletions, frameshift mutations, and the like.

In some embodiments, a single crRNA target site is chosen to introduce disruptions that render the Fel d 1 gene non-functional. For example, double-stranded breaks at a single target site introduced by Cas9 are repaired by non-homologous end joining (NHEJ), which can result in the insertion and deletion of a few base pairs which can introduce frameshift mutations which effectively knock-out the Fel d 1 gene by shifting the reading frame. In another example, a piece of DNA (“donor”) whose ends are identical to the region of interest (i.e. Feld 1 genomic sequence) is introduced, along with a sgRNA and Cas9 nuclease. As a result of repair by homology-directed repair (HDR), the donor DNA is introduced into the genome resulting in introduction of a foreign sequence into the Fel d 1 genomic sequence which effectively “knocks in” the foreign DNA. The foreign DNA sequence can be inserted so that the entire region is rendered non-functional, or that the foreign DNA sequence is expressed. The foreign DNA sequence may include, for example, an epitope tag (such as c-myc) or a reporter (such as green fluorescent protein (GFP)) and/or a non-functional Fel d 1 gene. Such procedures have been reviewed (see Wang et al., One-Step Generation of Mice Carrying Mutations in Multiple Genes by CRISPR/Cas-Mediated Genome Engineering, 153 (4): 910-918, 2013; Yang et al., One-Step Generation of Mice Carrying Reporter and Conditional Alleles by CRISPR/Cas-Mediated Genome Engineering, 154 (6): 1370-1379, 2013).

### Example: In Silico Identification of Guide RNA Target Sequences

A program that identifies guide RNA Target Sequences from a particular genomic sequence was used to identify a list of candidate guide RNA Target Sequences. The program is available on the Broad Institute website (http://www.broadinstitute.org/rnai/public/analysis-tools/sgrna-design). The genomic sequences of Fel d 1 chain 1 and chain 2, with 1 kb additional sequence on either side of them, were submitted separately to the program. For chain 1, the total length of the target sequence was 4516 base pairs (full sequence is set forth in SEQ ID NO: 1226), and for chain 2 the total length was 4416 base pairs (full sequence is set forth in SEQ ID NO: 1227). Additional parameters are provided in the table below (definitions can be found below):

TABLE 6

| | | | | | Off-Target | | |
|---|---|---|---|---|---|---|---|
| | | | Target | Initial | Match | | Off-Target |
| | Target | PAM | Window | Spacing | Ruleset | Off-Target | Match |
| Quota | Taxon | Policy | Policy | Requirement | Version | Tier Policy | Bin Policy |
| 8 | 9506 | NGG | 5-65 | 5 | 1 | 1 | 5.20.100 |

Quota = Desired number of candidate sgRNA sequences to pick for this target.
Target Taxon = Taxon of the target gene.
PAM Policy = Currently limited to NGG only.
Target Window Policy = Portion of the target region (as defined by Target Mode) which is preferentially targeted (given as a percent range, e.g. 5-65 means target the region between the 5th and 65th percentile of the target region).
Initial Spacing Requirement = When possible, pick sgRNA sequences which are separated by a certain distance (measured here in terms of percentage of the length of the entire target region). This is called “Initial” because, this requirement is relaxed in subsequent picking “rounds” if the quota is not met after examining all candidate sgRNAs for the target.
Off-Target Match Rule Set Version (“CFD score”) = Method of calculating an off-target match, or “CFD” (Cutting Frequency Determination) score; currently there is only one off-target rule set (“1”).
Off-Target Tier Policy = Method used to categorize off-target matches into “Tiers”; currently there is only one such policy (“1”), which breaks down as follows: Tier I: protein-coding regions; Tier II: any position within the transcribed sequence of a coding gene (intron or exon); Tier III: any position within the transcribed sequence of a non-coding gene; Tier IV: positions not contained in any gene (i.e. not transcribed).
Off-Target Match Bin Policy = Thresholds used to categorize off-target matches into “Match Bins” according to CFD score. There are four bins notated by three thresholds in increasing numerical order, separated by periods. Threshold values are in hundredths. For example, “5.20.100” represents the following 4 bins: Bin I: CFD = 1.0, Bin II: 1.0 > CFD ≥ 0.2, Bin III: 0.2 > CFD ≥ 0.05, Bin IV: 0.05 > CFD.

SEQ ID NOS: 1-1225 lists exemplary target sequences obtained from the above procedure. In general, the sequences were obtained from the corresponding chain (chain 1 or chain 2) and strand (sense or antisense) in which the target sequence is found, using a PAM Sequence of NGG, where N=A, C, G, or T. A total of 1428 target sequences were identified (795 for chain 1 and 633 for chain 2); however there were duplicates in this initial set so that 1225 of the 1428 are unique sequences and are set forth as SEQ ID NOS: 1-1225. With respect to the 1225 unique sequences, the following table represents the correspondence with SEQ ID NOS, Fel d 1 chain, strand, and the PAM sequence used to identify the sequences.

TABLE 7

| | | | PAM |
|---|---|---|---|
| Sequences | Chain | Strand | Sequence |
| SEQ ID NOS: 1-105 | 1 | sense | AGG |
| SEQ ID NOS: 106-123 | 1 | sense | CGG |
| SEQ ID NOS: 124-248 | 1 | sense | GGG |
| SEQ ID NOS: 249-323 | 1 | sense | TGG |
| SEQ ID NOS: 324-400 | 1 | antisense | AGG |
| SEQ ID NOS: 400-412 | 1 | antisense | CGG |
| SEQ ID NOS: 413-509 | 1 | antisense | GGG |
| SEQ ID NOS: 510-593 | 1 | antisense | TGG |
| SEQ ID NOS: 594-702 | 2 | sense | AGG |
| SEQ ID NOS: 703-718 | 2 | sense | CGG |
| SEQ ID NOS: 719-803 | 2 | sense | GGG |
| SEQ ID NOS: 804-881 | 2 | sense | TGG |
| SEQ ID NOS: 882-985 | 2 | antisense | AGG |
| SEQ ID NOS: 986-1008 | 2 | antisense | CGG |
| SEQ ID NOS: 1009-1118 | 2 | antisense | GGG |
| SEQ ID NOS: 1119-1225 | 2 | antisense | TGG |

Target sequences are located in the target DNA region and are referred to herein as the sgRNA Target Sequences. Corresponding crRNAs are identical in sequence to the target sequences. For example, appropriate crRNAs corresponding to the sgRNA Target Sequences provided in SEQ ID NOS: 1-1225 can include: AAAAAAAAAAUAAAGUGACC (SEQ ID NO: 1242) (crRNA encoded by target sequence of SEQ ID NO: 1); AAAAAUGACAGAAGAGGAUA (SEQ ID NO: 1243) (crRNA encoded by target sequence of SEQ ID NO: 2), etc.

Additionally, it is possible to rank the guide RNA target sequences according to an algorithm that predicts off-target and on-target effects. For example, algorithms can rank and pick candidate sgRNA sequences for the targets provided, while attempting to maximize on-target activity and minimizing off-target activity, if a reference genome is available. For example, the Broad Institute tool uses “Rule Set 2” (developed in conjunction with the Azimuth project at Microsoft Research) to model sgRNA on-target activity, and the CFD (Cutting Frequency Determination) score to evaluate off-target sites.

Additional tools for identifying guide RNA Target Sequences include those found at http://crispr.mit.edu/(see Hsu et al, DNA targeting specificity of RNA-guided Cas9 nucleases, Nature Biotechnology 31, 827-832 (2013)) and http://www.c-crisp.org/(see Heigwer, F., Kerr, G. & Boutros, M., E-CRISP: fast CRISPR target site identification. Nat. Methods 11, 122-123 (2014)).

### Example: Oligonucleotide Synthesis

Once guide RNA Target Sequences have been identified, DNA oligonucleotide sequences encoding guide RNAs and Cas9 may be synthesized. For example, the oligonucleotide sequences may be identical to any of the sgRNA Target Sequences set forth in SEQ ID NOS: 1-1225. In other embodiments, the oligonucleotide is at least 60%, 65%, 70%, 75%, 80%, 85%, 90%, or 95% identical to a sgRNA Target Sequence set forth in SEQ ID NOS: 1-1225. In specific aspects of the invention, the oligonucleotide encoding the crRNA portion of the guide RNA will be a 18-mer to 22-mer, such as a 20-mer, that is at least 60%, 65%, 70%, 75%, 80%, 85%, 90%, or 95% identical to the 20-mers set forth in SEQ ID NOS: 1-1225. Chemical synthesis of polynucleotides can be performed, for example, on commercial automated oligonucleotide synthesizers. Additionally, the oligonucleotide sequences may encode a tracrRNA portion. Examples of such tracrRNA-encoding oligonucleotides are set forth in SEQ ID NOS: 1228-1233. Further, oligonucleotides may be synthesized to encode any Cas9 protein or variant with dual or single endonuclease activity.

### Example: Cloning Into Vectors

General protocols for cloning include those found in Sambrook, Molecular Cloning: A Laboratory Manual, Cold Spring Harbor Press, Cold Spring Harbor, New York. For example, oligonucleotides encoding Cas9 and a pair of guide RNAs can be cloned into a single vector, or two separate vectors (one encoding Cas9 and one encoding the pair of guide RNAs). Protocols for such guide RNA and Cas9 cloning have been described (see Ran et al., 2013).

The vectors may be a plasmid or other recombinant vector which contains a promoter capable of expression of a transcript in mammalian cells, one or more cloning sites, a selectable marker for selection of transfected cells (e.g. Neomycin, Puromycin, Hydromycin, Zeocin/Bleo), and optionally a tag such as an epitope tag (e.g. FLAG, HA, Myc, 6xHis), protein purification tag (GST), or localization tag (GFP). Alternatively, the vectors may be viral vectors. Such viral vectors, and particularly retroviral vectors, have been used in a wide variety of gene delivery applications in cells, as well as living animal subjects. Viral vectors that can be used include, but are not limited to, retrovirus, adeno-associated virus, pox, baculovirus, vaccinia, herpes simplex, Epstein-Barr, adenovirus, geminivirus, and caulimovirus vectors. Additional examples of viral vectors include lentiviral vectors such as those based on HIV or FIV. Non-viral vectors include plasmids, liposomes, electrically charged lipids (cytofectins), DNA-protein complexes, and biopolymers. An example of a non-viral vector system is the Sleeping Beauty transposon system, which has been reviewed (see Aronovich, E. L., The Sleeping Beauty transposon system: a non-viral vector for gene therapy, Hum Mol Genet. 2011 Apr. 15; 20 (R1): R14-R20). In addition to a nucleic acid, a vector may also comprise one or more regulatory regions, and/or selectable markers useful in selecting, measuring, and monitoring nucleic acid transfer results (transfer to which tissues, duration of expression, etc.).

Examples of commercially-available mammalian expression vectors include p3xFLAG-CMV™ (Sigma Aldrich), p3xFLAG-Myc-CMV™ (Sigma Aldrich), pBI-CMV (Clontech), pcDNA™3.1 (Life Technologies), pCMV-Tag (Agilent Technologies), pIRES (Clontech), and others.

In aspects of the invention, vectors that may be used in the inventive methods may employ any of the following:
1) An RNA pol III promoter (such as a U6 promoter) for guide RNA expression.
2) A constitutive mammalian cell promoter for expression of Cas 9.
3) A nuclear localization signal (NLS) for directing Cas9 into the nucleus.
4) A Green-Fluorescent Protein (GFP) tag.
5) A tracrRNA sequence (gRNA scaffold).
6) A site for cloning a guide sequence (crRNA) oligonucleotide upstream of the tracrRNA sequence (G (N) 20 gRNA) by way of restriction enzyme digestion.
7) One or more selectable markers.

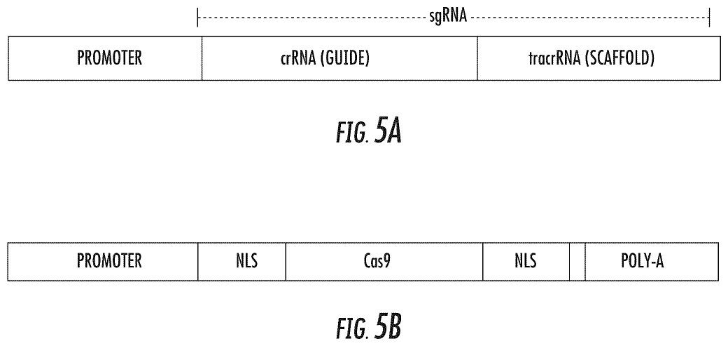

FIGS. 5A and 5B show examples of two basic expression cassettes for independent use or for incorporating into vectors. FIG. 5A shows an expression cassette comprising (from 5′ to 3′) a promoter and a chimeric DNA polynucleotide comprising a polynucleotide encoding a crRNA and a polynucleotide encoding a tracrRNA. Together the crRNA and tracrRNA make up the guide RNA that directs the Cas9 protein to a specific site in the genome. The promoter shown in FIG. 5A can be a pol III promoter such as a U6 promoter.

FIG. 5B shows an expression cassette comprising a promoter located 5′ of a polynucleotide encoding Cas9. The Cas9 encoding sequence is flanked by two nuclear localization signals (NLS). Additionally, the codons of the Cas9 encoding sequence can be optimized for expression in a species of interest (i.e. Felis catus cells). At the 3′ end of the expression cassette is a polynucleotide sequence encoding a poly-adenylation site. However, in other embodiments, such as use in lentiviral vectors, the nuclear localization signals may not be needed. One who is skilled in the art of molecular biology can customize the expression cassettes according to the requirements for a particular application. The promoter may be a mammalian promoter, an inducible promoter, a tissue-specific promoter, or the like.

Examples of animal and mammalian promoters known in the art include, but are not limited to, the SV40 early (SV40c) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, a baculovirus IE1 promoter, an elongation factor 1 alpha (EF1) promoter, a phosphoglycerate kinase (PGK) promoter, a ubiquitin (Ubc) promoter, an albumin promoter, the regulatory sequences of the mouse metallothionein-L promoter and transcriptional control regions, the ubiquitous promoters (HPRT, vimentin, beta-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis or disease related-promoters, and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo All control regions active in liver, alpha-fetoprotein gene control region active in liver, alpha 1-antitrypsin gene control region active in the liver, beta-globin gene control region active in myeloid cells, myelin basic protein gene control region active in oligodendrocyte cells in the brain, myosin light chain-2 gene control region active in skeletal muscle, and gonadotropic releasing hormone gene control region active in the hypothalamus, pyruvate kinase promoter, villin promoter, promoter of the fatty acid binding intestinal protein, promoter of the smooth muscle cell beta-actin, and the like. In addition, these expression sequences may be modified by addition of enhancer or regulatory sequences and the like.

Other types of promoters that may be used in the expression cassettes may include, without limitation, tissue-specific promoters, inducible promotors, light-regulated promoters, developmental-specific promoters, and the like.

As an example of tissue-specific promoters which may be useful according to the inventive methods, salivary gland-specific promoters such as parotid secretory protein (PSP) promoter, proline-rich protein (PRP) promoter, and salivary amylase promoter are described in Canadian Patent No. CA2369672C. Skin-specific promoters, such as keratin promoters for epidermal expression have also been described (see Blessing et al., Genes. Devel., 7, 204-15 (1993); Blessing et al., J. Cell. Biol., 135, 227-39 (1993); and Byrne et al., Mol., Cell. Biol., 13, 3176-90 (1993)). Other skin-specific promoters include E-cadherin, elastin or alpha-1 collagen promoters. These and other tissue-specific promoters known in the art may be useful for tissue-specific deletion of Fel d 1 expression.

Inducible promoters activate a level of gene expression that is dependent on the level of a ligand that is present to activate the promoter. Examples of inducible promoters are known in the art, and include those activated by tetracycline (TET-ON), rapamycin or its derivatives, or various steroidal ligands (e.g., glucocorticoid, estrogen, progestin, retinoid, or ecdysone) or analogs or mimetics thereof. Inducible promoter systems that are commercially available include the T-REX™ System (ThermoFisher Scientific, Waltham, MA) which is a mammalian expression system based on the binding of tetracycline to the Tet repressor and derepression of the promoter controlling expression of the gene of interest, as well as the Tet-On 3G Tetracycline-Inducible Gene Expression Systems (Clontech Laboratories, Inc., Mountain View, CA).

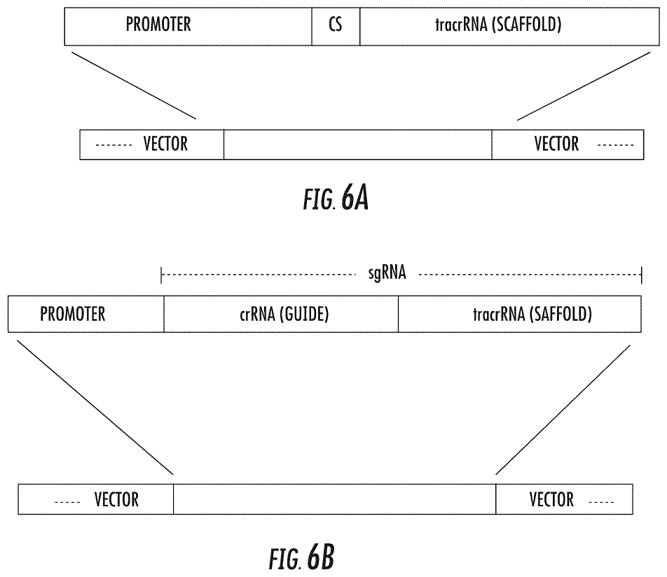

FIG. 6A shows a recombinant vector useful for cloning an oligonucleotide encoding a custom crRNA for expression of a guide RNA. The vector comprises (from 5′ to 3′) a promoter, a cloning site, and a polynucleotide encoding the tracr/scaffold portion of the guide RNA. For example, any of the 1225 polynucleotides set forth in SEQ ID NOS: 1-1225 can be synthesized as 20-mers and flanked with bases to match the restriction enzyme sites in the cloning site and cloned into the vector. FIG. 6B shows the vector of FIG. 6A with an oligonucleotide with a sequence chosen from one of the sequences set forth in SEQ ID NOS: 1-1225 cloned into the vector to complete the expression cassette.

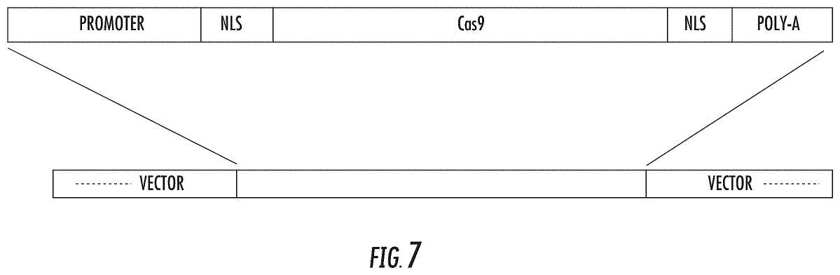

FIG. 7 shows a vector with the expression cassette of FIG. 5B.

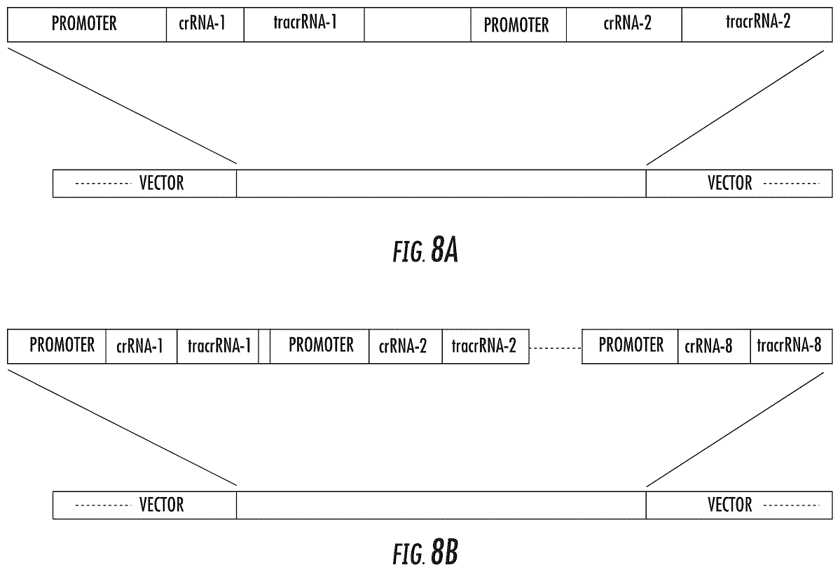

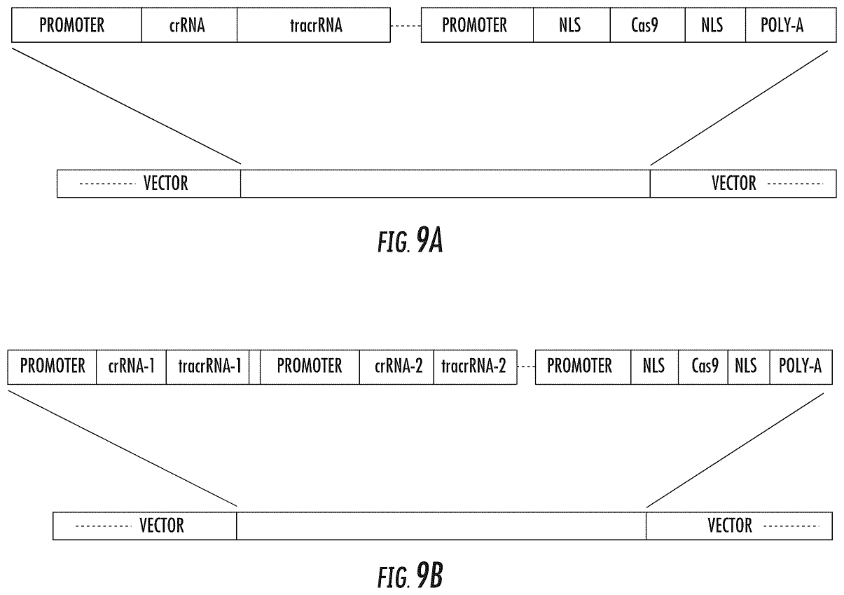

Additionally, vectors containing multiple expression cassettes are possible for multiplex gene targeting. For example, FIG. 8A shows a vector capable of expressing two guide RNAs, while FIG. 8B shows a vector capable of expressing up to 8 guide RNAs. Additionally, expression cassette encoding guide RNAs and Cas9 may be provided in separate vectors or the same vector. For example, FIG. 9A shows a vector capable of expressing a single guide RNA and Cas9, while FIG. 9B shows a vector capable of expressing a pair of guide RNAs and Cas9.

Additionally, a number of publicly-available vectors can be used for one or two-vector systems of guideRNA and Cas9 expression. Examples of commercially-available Cas9 vectors include pSpCas9 BB-2A-GFP (PX458), pLentiCRISPR v2, pLentiGuide-Puro, pGS-gRNA, pGS-gRNA-Neo, pAAV SpCas9 acceptor (PX552) (GenScript, Piscataway, NJ). Non-profit plasmid repositories such as Addgene are another source of vectors capable of expressing guideRNAs and Cas9.

### Example: Testing of Guide RNA Sequences

Guide RNA sequences may be tested for activity prior to testing in cell culture through routine methods. For example, in some embodiments, guide RNA sequences of interest are first synthesized by in vitro transcription. Next, individual guide RNA sequences may be tested in vitro to determine their efficacy. For example, in one embodiment, a target sequence may be synthesized and amplified by PCR to create a template. In a preferred embodiment, the genomic DNA target sequence of the particular cat cell line to be used in the implementation of the inventive methods is directly amplified by PCR to create the template. This ensures that the results would be representative of what would happen in the cell line of interest. The template may then be combined with a guide RNA of interest and a Cas9 nuclease. The efficiency with which Cas9 nuclease cleaves the template can then be measured using agarose gel electrophoresis. Such testing is available from commercially available kits (In Vitro Transcription and Screening Kits for sgRNA, Clontech Laboratories Inc, Mountain View, CA).

Alternatively or in addition, guide RNA sequences are tested in cell culture. Guide RNAs can be delivered to cells as either PCR amplicons containing an expression cassette or guide RNA-expressing plasmids. In addition to PCR and plasmid-based delivery methods, Cas9 and sgRNAs can be introduced into cells as mRNA and RNA, respectively. Alternatively, cultured cells can be transfected with Cas9 proteins and synthetic guide RNA oligonucleotides using conventional lipofection reagents. This generally involves: 1) assembly of a ribonuclear protein complex (RNP) by duplexing crRNA and tracrRNA oligos and mixing with Cas9 protein 2) reverse transfecting the RNP complex in cultured cells by way of transfection reagent.

For example, in one embodiment, 20-mer oligonucleotides encoding a pair of guide sequences may be synthesized into commercially available Cas9 vectors (all-in-one vector) and prepared for transfection into feline cell lines. Alternatively, the pair of guide sequences and Cas9 may be cloned into separate vectors (two-vector system). The following is an exemplary protocol for testing guide RNA sequences in cell culture.

Feline host cells are cultured in Eagle's Minimum Essential Medium supplied with fetal bovine serum to a final concentration of 10%. The cultures are incubated at 37° C. and subcultured when cell concentration is between 6 and 7×10⁴ cells/cm². The cells are then seeded 4-6×10⁴ cells/cm² in cell culture plate one day before transfection.

Guide RNA and Cas9 are introduced into feline host cells using standard methods. Vectors containing DNA encoding guide RNA and Cas9 may be introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter. Two to three days after transfection, the cell pool can be analyzed directly by Sanger sequencing, NGS (Next Generation Sequencing) and/or Surveyor assay. Sanger sequencing of the target region can detect overlapping peaks at close region of double strand break (DSB), if small insertion or deletion (indel) mutations are introduced. Surveyor assay (or T7E1 assay) uses enzymes of mismatch-specific DNA endonucleases to detect indel mutations at the targeted loci. By targeting and digesting mismatched heteroduplex double-strand DNA, this assay produces two or more smaller fragments, depending on number of mismatched sites on the region analyzed.

Transfected cells can be selected using antibiotic resistance or a GFP reporter if they are present on the Cas9 expression plasmid. Transfected cells (with or without selection) can be plated into 96 well plates at 1 cell/well density for cloning. This procedure can be also conducted using diluted host cell line on 10 cm plate to form colonies, which can be picked up and transferred to 24 well plate for future usage.

After expansion of the clones, the cells in each clone can be analyzed by Sanger sequencing to identify the clones harboring a mutation at the target region. Sequencing trace files will show overlapping peaks at regions where double strand breaks have been repaired by introducing small indels.

Alternatively, knock-out cell lines can be confirmed by Western Blot if a specific antibody is available, or through ELISA or other immunoassay.

Additionally, high throughput screening assays can be developed which assess guide RNA efficacy. Briefly, feline cells may be cultured in 96-well or 384-well plates. A library of sgRNA vectors may than be used to transfect the cells (two sgRNA sequences per well). After transfection the cells may be prepared for PCR based on amplification of an internal Fel d 1 sequence to confirm efficacy of each vector design, where successful knockout results in lack of amplification and vice versa. Alternatively or in addition, a cell line that expresses Fel d 1 can be used for the assay, and the cell media can be assayed for the Fel d 1 protein using an immunological-based assay such as a Radio Immune Assay (RIA) or ELISA.

### Creating Transgenic Cats

Once the efficacy of guide RNAs for Cas9-mediated knock out of Fel d 1 is confirmed, such guide RNAs can be used with Cas9 to create a transgenic cat. General procedures for gene targeting and animal cloning are available (see Denning C, and Priddle, H, New frontiers in gene targeting and cloning: success, application and challenges in domestic animals and human embryonic stem cells, Reproduction (2003) 126, 1-11; and Wang, B, and Zhou, J, Specific genetic modifications of domestic animals by gene targeting and animal cloning, Reproductive Biology and Endocrinology (2003), 1:103). The following Examples describe three different methodologies for creating Fel d 1 knockout cats.

### Example: Cloning a Genetically Engineered Fel d 1 Knockout Cat by Nuclear Transfer

The following Example is adapted from Shin et al., A cat cloned by nuclear transplantation: This kitten's coat-coloration pattern is not a carbon copy of its genome donor's, Nature, Vol 415 (2002), 859 and Supplementary Information.

### Oocyte Recovery and In Vitro Oocyte Maturation

Reproductive tracts from queens greater than 6 months of age are collected from routine ovariohysterectomy at local veterinary clinics. Ovaries are removed from the tract, rinsed in TL-Hepes, and then minced with a scalpel blade to release immature ova. For in vitro maturation, cat ova are then cultured in TCM 199 with Earle's salts supplemented with 0.36 mM pyruvate, 2.0 mM L-glutamine, 2.28 mM calcium lactate, 1.13 mM cysteine, 1% of a solution containing 10,000 U/ml Penicillin G, 10,000 μg/ml Streptomycin (P/S), 10 ng/ml EGF, 1 IU/ml hCG, 0.5 IU/ml cCG and 3 mg/ml fatty acid free BSA (IVM medium) for 24-30 hrs under 5% CO₂, 5% O₂, and 90% N₂ gas and humidified air atmosphere at 38° C.

### Enucleation

Following in vitro maturation, cumulus cells are removed from the ova by gently pipetting for 3 minutes in Hepes-buffered TCM 199 supplemented with 0.1% hyluronidase. After removal of cumulus cells, the oocytes are placed in a petri dish containing Hepes-buffered TCM 199 supplemented with 3 mg/ml fatty acid free BSA, 15.0 μg/ml cytochalasin B and 5 μg/ml Hoechst 33342, and enucleated using a beveled glass pipette mounted on Narshige micromanipulators while viewing with a Zeiss Microscope. Enucleation is confirmed by observation under UV light.

### Cell Culture and Preparation of Donor Cells

Skin cells or adult fibroblast cells from a donor cat are isolated and cultured in an appropriate mammalian cell culture medium, supplemented with 10% FBS for 2-5 days at 37° C. in an atmosphere of 5% CO₂ and air. The cells are then transfected with a vector comprising an expression cassette encoding Cas9 and a pair of guide RNAs (one designed to target the 5′ flanking region of chain 2, and one designed to target the 3′ flanking region of chain 1). sgRNA Target Sequences are for example chosen from those set forth in SEQ ID NOS: 1-1225. A sequencing assay or assay for Fel d 1 expression is performed on a portion of the cells to confirm knockout of Fel d 1 or a portion thereof. The cells are passaged multiple times then collected, frozen, and stored in liquid nitrogen. Three to 5 days prior to nuclear transfer the cell line is thawed and maintained in 4-well dishes (Nunc, Denmark) in DMEM/F12, supplemented with 10% FCS+1% P/S at 37° C. in an atmosphere of in 5% CO₂ and air.

### Nuclear Transfer, Electrofusion and Oocyte Activation

For nuclear transfer, the engineered Fel d 1 knockout donor cells are removed from the incubator, trypsinized using a 1% trypsin-EDTA solution, and placed into a petri dish containing Ca²⁺, Mg²⁺ free D-PBS with 0.3% BSA. Micromanipulation is then used to place a single nuclear donor cell into the perivitelline space of enucleated ova.

For electrofusion, the ovum/cell couplets are equilibrated in 0.3 M mannitol solution containing 0.1 mM Mg²⁺, then transferred to an electrofusion chamber containing the same medium. Cell fusion is induced by applying 2, 3.0 kV/cm 25 μsec DC pulses delivered by a BTX Electrocell Manipulator 200 (BTX, San Diego, CA). The couplets are then removed from the fusion chamber, washed and incubated in TCM 199 supplemented with 0.3% BSA and 5.0 μg/ml cytochalasin B, at 38° C. in and atmosphere of 5% CO₂ and air. Two hours after electrofusion, fused couplets are removed from the incubator and equilibrated in 0.3 mM mannitol containing 0.1 mM Ca²⁺ and 0.1 mM Mg²⁺, then placed into a fusion chamber containing the same medium and electropulsed by applying 2×, 1.0 KV/cm 50 μsec pulses, 5 seconds apart. The ova are then removed from the fusion chamber, washed, and incubated for 6-7 hrs in TCM 199 supplemented with 0.3% BSA, 10 μg/ml cycloheximide and 5 μg/ml cytochalasin B in a 5% CO₂, 5% O₂, 90% N₂ gas mixture in humidified air at 38° C. Cloned embryos are then cultured in modified Tyrode's solution supplemented with 0.36 mM pyruvate, 1.0 mM L-glutamine, 2.28 mM calcium lactate, 1% non-essential amino acids (NEAA) and 3 mg/ml fatty acid free BSA (IVC 1 medium) for 1-3 days.

### Synchronization of Recipient Females and Embryo Transfer

Cloned embryos are surgically transferred into the oviducts of recipient queens. Estrus synchronization of recipient queens is attained using the same hormone injection regimen described above. Following embryo transfer, transabdominal ultrasonography is utilized to monitor for pregnancy.

Cloned kittens are reared to 6 months of age. Samples of saliva are tested for the production of Fel d 1 according to an ELISA assay. Additionally, kittens are anesthetized and a biopsy of the salivary gland is taken. Salivary gland explants are grown in organ culture dishes in DMEM with 10% FCS on filter paper. The explants are subject to genomic DNA extraction as described above and PCR and sequence analysis. ELISA testing of saliva samples shows no detectable Fel d 1 in comparison to controls, and PCR and sequencing analysis confirm complete disruption of chain 1 and chain 2 Fel d 1 genomic DNA.

Alternatively or in addition to ELISA testing, Western Blot Analysis can be used to confirm lack of Fel d 1 production in saliva or explant samples.

### Example: Cloning a Genetically Engineered Fel d 1 Knockout Cat by Direct Gamete Genetic Modification

The following Example is adapted from Wongsrikeao et al., Antiviral restriction factor transgenesis in the domestic cat, Nat Methods.; 8 (10): 853-859 (2011), incorporated by reference herein in its entirety. The skilled artisan can supplement this Example with specific details provided in the Wongsrikeao manuscript and Supplemental Materials.

Gametes from both domestic sexes are obtained without added animal procedures by micro-dissecting gonads discarded after spaying or neutering. In vitro matured grade I and II domestic cat oocytes are subjected to perivitelline space microinjection (PVSMI) with 100 picoliters of concentrated lentiviral vector. The lentiviral vector includes expression cassettes for two guide RNAs (one designed to target the 5′ flanking region of chain 2, and one designed to target the 3′ flanking region of chain 1) as well as Cas9. Injection is timed 10-12 h before or 10-12 hr after in vitro fertilization (IVF) with feline sperm. The embryos are then cultured and selected for implantation from cleaved, post-IVF oocytes and are transferred into surgically exposed fallopian tubes at 48-72 hours after lentiviral vector transduction. The transfers are performed in hormonally synchronized queens prepared by a 14/10 hour light/dark environment. Queens are administered PMSG on day-4, and HCG on day-1 with respect to lentiviral vector transduction and are mated ad lib from the day of HCG injection until the day before embryo transfer with a vasectomized, azoospermia-verified tomcat to induce ovulation and corpus luteum formation. At surgery follicles are punctured with a needle if not naturally ovulated. The implanted embryos are allowed to develop in utero. Skin and salivary gland samples from kittens are assessed for Fel d 1 protein expression and genomic deletion. The results show complete knockout of Fel d 1, and complete lack of production of this allergen.

### Example: Cloning a Genetically Engineered Fel d 1 Knockout Cat by Embryonic Stem Cell Manipulation

Embryonic stem cells of a cat are cultured and transfected with a vector comprising an expression cassette encoding Cas9 and a pair of guide RNAs (one chosen to target an interior region of chain 2, and one chosen to target a 3′ flanking region of chain 1). Sequencing analysis may be performed on a sample of the culture to confirm knockout of all or a portion of a Fel d 1 genomic sequence. The embryonic stem cells may then be transferred to a blastocyst by aggregation or injection. Manipulated embryos are then transferred to a pseudopregnant female recipient cat and allowed to mature. The resulting offspring are heterozygotes for the Fel d 1 knockout. Heterozygotes are then cross-bred to produce homozygotes for the Fel d 1 knockout.

Detailed procedures for embryonic stem cell derivation, culture, manipulation, and implantation are known in the art and are described in U.S. Patent Application Publication No. 2003/0177512, incorporated by reference. The skilled artisan can supplement this Example with specific details provided in the '512 Patent Application Publication.

### Example: Treatment of Salivary Glands of an Adult Wild-Type Cat

An FIV-based vector is genetically engineered to include expression cassettes encoding Cas9 and a pair of guide RNAs selected to target two of the guide RNA target sequences set forth in SEQ ID NOS: 1-1225. The guide RNAs are selected to target flanking regions of Fel d 1 sequences (e.g., one upstream of chain 2, one downstream of chain 1). The expression cassettes are driven by a salivary-gland specific promoter (salivary amylase promoter) to minimize the potential for systemic effects. Viral particles are produced by the transfection of 293 T cells with transfer vector, packaging vector, and envelope vector. Supernatants are collected 48 and 72 h post-transfection. Viral particles are concentrated 100-fold from pooled supernatants spun in an ultracentrifuge resuspended in PBS. Viral titers of the FIV-based vector are determined by FACS analysis of 293 T cells infected with diluted viral supernatant.

A 12 month-old male domestic cat is given a combination of Dexmedetomidine (0.025-0.0375 mg/kg IM)+Butorphanol (0.4-0.6 mg/kg IM)+Ketamine (5-7.5 mg/kg IM). The cat's salivary glands are cannulated through their ducts using an extended polyethylene tube. The cannulated glands are repeatedly inoculated with the FIV-based vector. The cannulas are then removed and the cat is allowed to recovery from the procedure.

At 1 week, 2 weeks, and 1 month post-procedure, samples of saliva are tested for the production of Fel d 1 according to Western Blot Analysis. Additionally, the cat is re-anesthetized and a biopsy of the salivary gland is taken. Salivary gland explants are grown in organ culture dishes in DMEM with 10% FCS on filter paper. The explants are subject to genomic DNA extraction as described above and PCR and sequencing analysis. Western blot analysis of saliva samples show no Fel d 1 immunoreactivity in comparison to controls, and PCR sequencing analysis, or Surveyor Assay to confirm complete excision of chain 1 and chain 2 Fel d 1 genomic DNA.

### Example: Treatment of Skin of an Adult Wild-Type Cat

An FIV-based vector is genetically engineered to include expression cassettes encoding Cas9 and a pair of guide RNAs that are selected to target internal regions of Fel d 1 sequences (e.g., one internal to chain 1 and one internal to chain 2). The guide RNAs, and in particular the crRNA portion of the sgRNA, can be chosen based on the sgRNA Target Sequences provided in SEQ ID NOS: 1-1225. The expression cassettes are driven by a skin-specific promoter (keratin promoter) to minimize the potential for systemic effects. Viral particles of the FIV-based vector are produced as described in the previous example. The FIV-based vector is then injected intradermally into a 3 year-old female domestic cat using a 34 gauge needle. Alternatively, the cat is shaved to expose the skin and the FIV-based vector of the previous example is topically applied in a formulation that includes a permeation enhancer such as alcohols (e.g., methanol), alkyl methyl sulfoxides (e.g., DMSO), pyrrolidones (e.g., 2-pyrrolidone), surfactants, urea, glycerol monolaurate, polyethylene glycol monolaurate, glycerol monolaurate, docainehydrochloride, hydrocortisone, menthol, or methyl salicylate. Optionally, the skin is subject to reversible electroporation to enhance uptake of the vector according to established methods, such as those described in U.S. Pat. No. 6,697,669, incorporated by reference.

At 1 week, 2 weeks, and 1 month post-procedure, samples of skin are biopsied and subject to analysis of Feld 1 expression or genomic deletion as described in the previous example. The results show no Fel d 1 immunoreactivity in comparison to controls and confirm excision of Feld 1 genomic DNA at the target sequences.

### Example: Systemic Treatment of a Juvenile Wild-Type Cat

A catheter is placed in the cephalic vein of a 6 month old cat using a 22 gauge needle. An FIV-based vector is genetically engineered to include expression cassettes encoding Cas9 and a pair of guide RNAs selected to target two of the guide RNA target sequences set forth in SEQ ID NOS: 1-1225. The guide RNAs are selected to target flanking regions of Fel d 1 sequences (e.g., one upstream of chain 2, one downstream of chain 1). Viral particles of the FIV-based vector are produced as described previously. The expression cassettes for Cas9 are optionally driven by promoters for keratin and amylase, which limit expression to skin and salivary glands, respectively. The FIV-based vector is administered intravenously once a day for 5 consecutive days. At 1 month, 3 months, and 6 months post-procedure, samples of skin and salivary gland are biopsied and subject to analysis of Fel d 1 expression or genomic deletion as described in the previous examples. The results show no Fel d 1 immunoreactivity in comparison to controls and confirm complete excision of chain 1 and chain 2 Fel d 1 genomic DNA.

### Example: Inactivation of Fel d 1 by Knock-In of Green Fluorescent Protein

A plasmid donor (400 ng), with two homology arms on each side flanking a green fluorescent protein (GFP) reporter is introduced into cat zygotes along with a vector encoding Cas9 and a guide RNA. Each of the homology arms is around 800 bp. The sgRNA and plasmid donor are designed to insert the GFP reporter in place of the Fel d 1 promoter and the Chain 1 and Chain 2 start codons. The plasmid donor would be used as a template in the process of HDR for scaling the double-stranded break in the DNA. In this way, the coding sequence of GFP in between the homology arms would take the place of the promoter region and start codons, when the break is repaired. A stop codon is included in the plasmid donor after the GFP reporter gene sequence so that only the GFP sequence is expressed. After injection of the vectors into zygotes, in vitro differentiated blastocysts are explanted into culture to derive embryonic stem cells. PCR genotyping and sequencing is used to identify embryonic stem cell lines carrying a correctly targeted insert. GFP expression is confirmed via a microplate fluorescence reader using appropriate excitation and emission filter settings. Cat zygotes with the correct insertion are then allowed to develop into embryos, which are implanted into recipient female cats and allowed to develop to term. The live-born kittens are confirmed to express GFP and also lack Fel d 1 expression as described in previous examples.

### Example: Confirmation of Fel d 1 Hypoallergenicity

Allergy testing can be performed on a group of confirmed human Fel d 1 allergy sufferers to confirm success of Fel d 1 knockout procedures in producing a Fel d 1 hypoallergenic phenotype. For example, cat hair or saliva may be collected from a Fel d 1 knockout cat and an extract of the hair or saliva may be prepared and used as an inoculant in skin testing procedures (skin prick, intradermal, or skin patch testing). The results can be compared to a standardized cat hair extract indicated in skin testing diagnosis such as Greer's Standardized Cat Hair (GREER®, Lenoir, NC). Lack of an allergic response (e.g., swelling, redness, hives, sneezing, breathing difficulties, asthma, itchy skin, runny nose, stuffy nose, coughing, and/or eye irritation) can confirm that the particular Fel d 1 knockout results in a hypoallergenic phenotype.

Alternatively or in addition, serum samples from confirmed human Fel d 1 allergy sufferers may be collected and various in vitro immunoassays measuring IgE reactivity as well as allergenic activity known in the art may be performed using the aforementioned inoculants as test samples. For example, the ability of such test samples to inhibit the binding of Fel d 1 protein to IgE antibodies can be tested by way of a radioallergosorbent test (RAST) inhibition assay. Also, the activity of such test samples in a histamine release assay using the stripped basophil technique can be assessed and compared to that of Fel d 1 (see Chapman et al., The European Union CREATE Project: A model for international standardization of allergy diagnostics and vaccines, J Allergy Clin Immunol, 122 (5): 882-889 (2008)).

The present invention has been described with reference to particular embodiments having various features. In light of the disclosure provided above, it will be apparent to those skilled in the art that various modifications and variations can be made in the practice of the present invention without departing from the scope or spirit of the invention. For example, while the present disclosure, in embodiments, discusses genomic editing in Felis catus (the domestic house cat), the present invention contemplates compositions and methods for removal of Fel d 1 from any wild or domesticated feline, including those of genera Acinonyx, Caracal, Calopuma, Felis, Leopardus, Leplailurus, Lynx, Olocolobus, Pardofelis, Prionailurus, and Profelis. Additionally, one skilled in the art can recognize that the methods provided in this disclosure may be modified to target and remove other cat allergens. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1-20. (canceled)

21. A method of editing a Fel d 1 gene in an isolated Felis catus cell, the method comprising:
introducing i) one or more gRNAs and ii) Cas9 into an isolated Felis catus cell and editing an endogenous Fel d 1 gene of the Felis catus cell in a manner to result in impaired or absent Fel d 1 protein expression;
wherein the gRNA are at least 60% identical to gRNA of SEQ ID NOS: 1244 to 2468.

22. The method of claim 21, wherein:
the gRNA are at least 75% identical to gRNA of SEQ ID NOS: 1244 to 2468.

23. The method of claim 22, wherein:
the gRNA are at least 90% identical to gRNA of SEQ ID NOS: 1244 to 2468.

24. The method of claim 21, wherein:
the gRNA are chosen from SEQ ID NOS: 1244 to 2468.

25. A genetically engineered Felis catus cell line:
comprising a Fel d 1 genomic sequence lacking at least a portion of the Fel d 1 genomic sequence of a cell line from which the engineered cell line is derived, wherein the portion lacking is a sequence at least 60% identical to any of SEQ ID NOS: 1-1225.

26. The genetically engineered Felis catus cell line of claim 25 comprising reduced or absent Fel d 1 protein expression.

27. The genetically engineered cell line of claim 26, wherein the reduced or absent Fel d 1 protein expression is hypoallergenic in humans.

28. A genetically engineered cat comprising the genetically engineered cell line of claim 25.

29. The genetically engineered cat of claim 28 comprising a Fel d 1 gene with reduced or absent Fel d 1 protein expression.

30. The genetically engineered cat of claim 29, wherein the reduced or absent Fel d 1 protein expression is hypoallergenic in humans.

31. The genetically engineered cat of claim 29, wherein the Fel d 1 protein expression is reduced or absent in skin, salivary glands, perianal glands, or lachrymal glands.

32. The genetically engineered cat of claim 28, wherein all or a portion of the Fel d 1 genomic sequence of the genetically engineered cat is deleted from somatic cells.

33. F1 progeny of a male and female cat of claim 28.

34. A method of deleting a genomic sequence from the genome of a Felis catus cell, comprising:
introducing guide RNA into a Felis catus cell and using a CRISPR system directing a double stranded break in the genome of the Felis catus cell, wherein at least a portion of a Fel d 1 gene is removed resulting in impaired or an absence of Fel d 1 protein expression.

35. The method of claim 34, wherein the portion of the Fel d 1 gene removed represents a conserved region identified from genomic sequencing data.

36. The method of claim 35, wherein the genomic sequencing data is obtained from genomic DNA isolated from feline tissue samples.
